SEQUENCE LISTING (3 sequences)

SEQ ID NO: 1 - DNA, 30 bp, synthetic construct
  source 1..30: mol_type = other DNA
ggaggaaaaactgtttcatacagaaggcgt

SEQ ID NO: 2 - DNA, 20 bp, synthetic construct
  source 1..20: mol_type = other DNA
cttcaagagcaacagtgctg

SEQ ID NO: 3 - DNA, 20 bp, synthetic construct
  source 1..20: mol_type = other DNA
aggtcgctgtgtttgagcca

(19) United States
(12) Patent Application Publication
(10) Pub. No.: US 2025/0177467 A1
(43) Pub. Date: Jun. 5, 2025

(54) COMPOSITIONS AND METHODS FOR MODULATING NUCLEAR FACTOR OF ACTIVATED T-CELLS (NFAT) ACTIVITY OF T CELLS

(71) Applicant: Yale University, New Haven, CT (US)

(72) Inventors: Yung-Chi Cheng, New Haven, CT (US); Wing Lam, New Haven, CT (US)

(21) Appl. No.: 18/842,269

(22) PCT Filed: Feb. 28, 2023
(86) PCT No.: PCT/US2023/014151

§ 371 Date: Aug. 28, 2024

Related U.S. Application Data
(60) Provisional application No. 63/315,163, filed on Mar. 1, 2022.

(51) Int. Cl. A61K 36/539; A61K 31/12; A61K 31/352; A61K 31/37; A61K 31/7034; A61K 31/7048; A61K 36/484; A61K 36/65; A61K 36/725; A61K 39/395

(52) U.S. Cl. CPC A61K 36/539; A61K 31/12; A61K 31/352; A61K 31/37; A61K 31/7034; A61K 31/7048; A61K 36/484; A61K 36/65; A61K 36/725; A61K 39/3955

(57) ABSTRACT

The present invention relates to methods for modulating NFAT activity and/or restoring the suppressed Nuclear Factor of Activated T-cells (NFAT) activity in T cells of a subject in need thereof. In another aspect, the invention provides different herbal compositions for modulating NFAT activity in T cells of a subject in need thereof.

### CROSS-REFERENCE TO RELATED APPLICATIONS

The present application claims priority under 35 U.S.C. § 119 (e) to U.S. Provisional Application No. 63/315,163, filed Mar. 1, 2022, which is hereby incorporated by reference herein in its entirety.

### SEQUENCE LISTING

The present application contains a Sequence Listing in XML format and is herein incorporated by reference in its entirety. Said XML file, created on Feb. 15, 2023, is named 047162_7355WO1_SequenceListing.xml and is 3000 bytes in size.

### BACKGROUND OF THE INVENTION

Nuclear factor of activated T-cells (NFAT) is a family of transcription factors that consists of five members—NFATc1, NFATc2, NFATc3, NFATc4, and NFAT5. NFAT is well-known for modulating immune responses. At least one member of NFAT family is expressed in most cells of the immune system. Additionally, NFAT is also known to be involved in the development of cardiac, skeletal muscle, and nervous systems. NFAT is being investigated as a drug target for several different diseases and disorders such as, for example, inflammatory bowel diseases and several types of cancer. Also, there is an unmet need in the art to develop compositions and methods for modulating NFAT activity. The present invention addresses this need.

### SUMMARY OF THE INVENTION

In one aspect, the invention provides a method of enhancing Nuclear Factor of Activated T-cells (NFAT) activity and/or restoring the suppressed NFAT activity in a subject in need thereof, wherein the method comprises administering to the subject a therapeutically effective amount of least one herbal composition selected from a group of herbal compositions as described elsewhere herein.

In another aspect, the invention provides a method of enhancing efficacy of Chimeric Antigen Receptor T-cell (CAR T) immunotherapy in a subject, wherein the method comprises administering to the subject a therapeutically effective amount of least one herbal composition selected from the group of herbal compositions as described elsewhere herein.

In yet another aspect, the invention provides a method of enhancing efficacy of immune checkpoint blockade therapy in a subject, wherein the method comprises administering to the subject a therapeutically effective amount of least one herbal composition selected from the group of herbal compositions as described elsewhere herein.

In yet another aspect, the invention provides an herbal composition for modulating NFAT activity in T cells of a subject in need thereof, wherein the herbal composition is selected from the group of compositions as described elsewhere herein.

In yet another aspect, the invention provides a method of enhancing effectiveness of a vaccine in a subject, wherein the method comprises administering to the subject a therapeutically effective amount of least one herbal composition selected from the group of herbal compositions as described elsewhere herein.

In certain embodiments, the herbal composition is at least one selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof;
(f) an herbal extract of P, a fraction thereof or any active chemical present in the herbal extract or the fraction thereof, and
(g) an herbal extract comprising an extract of P, a fraction thereof or any active chemical present in the herbal extract or the fraction thereof.
(h) an herbal extract of Z, a fraction thereof or any active chemical present in the herbal extract or fraction thereof; and
(i) an herbal extract comprising an extract of Z, a fraction thereof or any active chemical present in the herbal extract or fraction thereof.

In certain embodiments, the active chemical in the herbal extract of S or the fraction thereof is at least one selected from the group consisting of oroxylin A 7-O-beta-D-glucuronide, baicalein, oroxylin A, baicalin, isomer of baicalin, chrysin 6-C-arabinoside 8-C-glucoside, acteoside, viscidulin I 2′-O-glucoside, and chrysin.

In certain embodiments, the active chemical in the herbal extract of G or the fraction thereof is at least one selected from the group consisting of liquiritigenin, isoliquiritigenin, liquiritin, isoliquiritin, licochalcone A, and glycycoumarin.

In certain embodiments, the active chemical in the herbal extract of P or the fraction thereof is at least one selected from the group consisting of gallic acid, 4-o-methyl paeoniflorin, 4-o-methyl paeoniflorin isomer, 6′-O-galloyl paeoniflorin, albiflorin, and paeoniflorin.

In certain embodiments, the herbal composition is administered to the subject orally.

In certain embodiments, the herbal composition is formulated as one selected from the group consisting of a pill, a tablet, a capsule, soup, tea, a concentrate, a dragee, a liquid, a drop, and a gelcap.

In certain embodiments, the therapeutically effective amount of the herbal composition is about 20 mg/day to about 2 g/day.

In certain embodiments, the therapeutically effective amount of the herbal composition is about 1,600 mg/day.

In certain embodiments, the method induces protein phosphorylation of T cell receptor (TCR) downstream cascades and inhibits SH2-containing phosphatase 2 (SHP2) activity.

In certain embodiments, the herbal composition is administered at a time selected from prior to, simultaneously with, and after administration of an immune checkpoint inhibitor therapy or a Chimeric Antigen Receptor T (CAR T) cell therapy or a vaccine.

In certain embodiments, the immune checkpoint inhibitor therapy includes administering at least one immune checkpoint inhibitor selected from the group consisting of Ipilimumab, Pembrolizumab, Nivolumab, Durvalumab, Avelumab, Dostarlimab-gxly, Cemiplimab-rwlc, Toripalimab, Camrelizumab, Tislelizumab, Penpulimab, Zimberelimab, Sintilimab, and Atezolizumab.

In certain embodiments, the method is advantageous for at least one selected from the group consisting of:
a) enhancing efficacy of the CAR T cell therapy,
b) enhancing efficacy of the immune checkpoint inhibitor therapy, and
c) enhancing effectiveness of the vaccine.

In certain embodiments, the method is used for cancer chemoprevention.

In certain embodiments, the composition further comprises a pharmaceutically acceptable carrier.

In certain embodiments, the subject is a mammal. In certain embodiments, the subject is a human subject.

### BRIEF DESCRIPTION OF THE DRAWINGS

The following detailed description of specific embodiments of the invention will be better understood when read in conjunction with the appended drawings. For the purpose of illustrating the invention, there are shown in the drawings specific embodiments. It should be understood, however, that the invention is not limited to the precise arrangements and instrumentalities of the embodiments shown in the drawings.

FIGS. 1A-1F illustrate effects of YIV-906 and its components herbs: Glycyrrhiza uralensis Fisch (G), Paeonia lactiflora Pall (P), Scutellaria baicalensis Georgi (S), and Ziziphus jujuba Mill (Z) on NFAT mediated transcriptional activity of Jurkat cells incubated with wild type Raji cells without or with staphylococcal superantigen E (SEE). The effects of YIV-906 and its single herbs on NFAT mediated transcriptional activity of Jurkat cells incubated with Raji cells without SEE (FIG. 1A) and with SEE (FIG. 1B) are compared. Further, the effects of two herbs combination on NFAT mediated transcriptional activity of Jurkat cells incubated with Raji cells without SEE (FIG. 1C) and with SEE (FIG. 1D) are also shown. Additionally, the effects of deletion of one herb of YIV-906, -G, -P, -S and -Z on NFAT mediated transcriptional activity of Jurkat cells incubated with Raji cells without SEE (FIG. 1E) and with SEE (FIG. 1F) are also shown. Equivalent amount of water extracts up to 320 ug/ml was added to Jurkat cells, which were stably transfected with NFAT luciferase reporter, together with Raji cells without or with staphylococcal superantigen E (SEE) 10 ng/ml for 24 h before luciferase activity was measured.

FIGS. 2A-2F illustrate effects of YIV-906 and its components herbs: Glycyrrhiza uralensis Fisch (G), Paeonia lactiflora Pall (P), Scutellaria baicalensis Georgi(S), and Ziziphus jujuba Mill (Z) on NFAT mediated transcriptional activity of PD1 overexpressed Jurkat cells incubated with PD-L1 overexpressed Raji cells without or with SEE. The effects of YIV-906 and its single herbs on NFAT mediated transcriptional activity of Jurkat-PD1 cells incubated with Raji-PD-L1 cells without SEE (FIG. 2A) and with SEE (FIG. 2B) are shown. The effects of two herbs combination on NFAT mediated transcriptional activity of Jurkat-PD1 cells incubated with Raji-PD-L1 cells without SEE (FIG. 2C) and with SEE (FIG. 2D) are also shown. Additionally, the effects of deletion of one herb of YIV-906, -G, -P, -S and -Z on NFAT mediated transcriptional activity of Jurkat-PD1 cells incubated with Raji-PD-L1 cells without SEE (FIG.

2E) and with SEE (FIG. 2F) are shown. Equivalent amount of water extracts up to 320 ug/ml was added to Jurkat-PD1 cells, which were stably transfected with NFAT luciferase reporter, together with Raji-PD-L1 cells without or with SEE 10 ng/ml for 24 h before luciferase activity was measured.

FIGS. 3A-3D are graphs showing effects of YIV-906 on T activation marker, CD69 of PD1 overexpressed Jurkat cells when incubated Raji without or with PD-L1 over-expression. Effects of YIV-906 on the median fluorescence (FIG. 3A) and population (FIG. 3B) of Jurkat PD1 cells when incubated with wild type Raji cells in absence or presence of SEE antigen are shown. Effects of YIV-906 on the median fluorescence (FIG. 3C) and population (FIG. 3D) of Jurkat PD1 cells when incubated with PD-L1 overexpressed Raji cells in absence or presence of

SEE antigen are also shown. Water extract of YIV-906 up to 320 ug/ml was added to Jurkat-PD1 cells together with wild type or PD-L1 overexpressed Raji cells without or with SEE 3 ng/ml, 10 ng/ml for 24 h. The expression of CD69 was determined by flow cytometry in which FITC conjugated anti-CD69 was used to determine the expression of CD69 protein on the membrane of Jurkat cells and PE conjugated anti-PD1 was used to gate Jurkat PD1 cell from the mixture of Jurkat/Raji cells.

FIGS. 4A-4D show chemical structures of the pure compounds selected from YIV-906 competent herbs: S (FIG. 4A), P (FIG. 4B), Z (FIG. 4C) and G (FIG. 4D) for examining their effect on NFAT activity of Jurkat cells.

FIGS. 5A-5D illustrate effects of S compounds on NFAT mediated transcriptional activity of PD1 overexpressed Jurkat cells incubated with PD-L1 overexpressed Raji cells without or with SEE. The effects of S compounds on NFAT mediated transcriptional activity of Jurkat-PD1 cells incubated with wild type Raji without SEE (FIG. 5A) and with SEE (FIG. 5B) are shown. The effects of S compounds on NFAT mediated transcriptional activity of Jurkat-PD1 cells incubated with Raji-PD-L1 cells without SEE (FIG. 5C) and with SEE (FIG. 5D) are also shown. S compounds, up to 40 uM was added to Jurkat-PD1 cells, which were stably transfected with NFAT luciferase reporter, together with wild type Raji cells or Raji-PD-L1 cells without or with staphylococcal superantigen E (SEE) 10 ng/ml for 24 h before luciferase activity was measured.

FIGS. 5E-5H illustrate effects of P compounds on NFAT mediated transcriptional activity of PD1 overexpressed Jurkat cells incubated with PD-L1 overexpressed Raji cells without or with staphylococcal superantigen E (SEE). The effects of P compounds on NFAT mediated transcriptional activity of Jurkat-PD1 cells incubated with wild type Raji without SEE (FIG. 5E) and with SEE (FIG. 5F) are shown. The effects of P compounds on NFAT mediated transcriptional activity of Jurkat-PD1 cells incubated with Raji-PD-L1 cells without SEE (FIG. 5C) and with SEE (FIG. 5H). P compounds, up to 160 uM was added to Jurkat-PD1 cells, which were stably transfected with NFAT luciferase reporter, together with wild type Raji cells or Raji-PD-L1 cells without or with SEE 10 ng/ml for 24 h before luciferase activity was measured.

FIGS. 6A-6D illustrate effects of G and Z compounds on NFAT mediated transcriptional activity of PD1 overexpressed Jurkat cells incubated with wild type Raji or PD-L1 overexpressed Raji cells without or with (SEE. The effects of G and Z compounds on NFAT mediated transcriptional activity of Jurkat-PD1 cells incubated with wild type Raji without SEE (FIG. 6A) and with SEE (FIG. 6B) are shown. The effects of S compounds on NFAT mediated transcriptional activity of Jurkat-PD1 cells incubated with Raji-PD-L1 cells without SEE (FIG. 6C) and with SEE (FIG. 6D) are also shown. G and Z compounds, up to 40 uM, was added to Jurkat-PD1 cells, which were stably transfected with NFAT luciferase reporter, together with wild type Raji cells or Raji-PD-L1 cells without or with staphylococcal superantigen E (SEE) 10 ng/ml for 24 h before luciferase activity was measured.

FIGS. 7A-7F illustrate effects of YIV-906, S and S compounds on NFAT mediated transcriptional activity of Jurkat-PD1 cells and T cell receptor-αβ (TCRαβ) knockout Jurkat-PD1 cells. Detection of TCRαβ of Jurkat-PD1 (FIG. 7A) and Jurkat-PD1 TCRαβ knockout cells (FIG. 7B) by using FITC conjugated anti-TCRαβ and flowcytometry is presented. Effects of YIV-906 (FIG. 7C), S (FIG. 7C) and S compounds (FIG. 7D) on NFAT mediated transcriptional activity of Jurkat-PD1 cells are shown. Effects of YIV-906 (FIG. 7E), S (FIG. 7E) and S compounds (FIG. 7F) on NFAT mediated transcriptional activity of Jurkat-PD1 TCRαβ knockout cells are also shown.

FIGS. 8A-8D are western blot analyses showing effects of YIV-906 (FIG. 8A), single herbs (FIG. 8A), the fractions of YIV-906 (FIG. 8B), P (FIG. 8C), and S (FIG. 8D) on protein phosphorylation of T cell receptor signaling cascades including, Lck, Fyn, Zap70, LAT and Pyk2. C18 solid phase extract column was used to fractionize YIV-906, S and P by eluting with water and increase percentage of acetonitrile/methanol (A/M). All eluted fractions were dried and reconstituted with water to equivalent concentration at 100 mg/ml. Jurkat cells were treated with YIV-906 (FIG. 8A), single herbs (FIG. 8A), the fractions of YIV-906 (FIG. 8B), P (FIG. 8C), and S (FIG. 8D) at equivalent dose 320 ug/ml for 45 min. Western blot analysis was used to determine the protein phosphorylation using specific antibodies. GAPDH was used to normalize the protein loading.

FIGS. 9A-9B show chemical identification of fractions of P (FIG. 9A) and S (FIG. 9B) after C18 column fractionation and their impacts on protein phosphorylation for Lck, Zap70, Lat, Fyn/Src, Pyk2. Equivalent concentration, 10 mg/ml, was used for LC-MS analysis. Equivalent concentration, 320 ug/ml for 45 min, was used for treating Jurkat cells for western blotting analysis. Identified chemicals (putative), based on their mass and mass of their fragments, were listed under each fraction. Non-identified chemical was not listed in this figure. Fraction could induce phosphorylation of specific protein was indicted as red upward arrow.

FIG. 10 show effects of S and P compounds on protein phosphorylation of T cell receptor signaling cascades including, Lck, Fyn, Zap70, LAT and Pyk2. Jurkat cells were treated with S and P compounds for 45 min. Western blot analysis was used to determine the protein phosphorylation using specific antibodies.

FIGS. 11A-11D illustrate effects of YIV-906 and its components on the SHP2 activity. FIG. 11A show effects of YIV-906 without or with beta-glucuronidase treatment on the SHP2 activity. FIG. 11B show effects of YIV-906 and its component herbs: G, P, S and Z at 1 mg/ml on the SHP2 activity. FIG. 11C show effects of G compounds at 40 uM on the SHP2 activity. FIG. 11D show effects of S compounds at 40 uM on SHP2 activity. Activity of SHP2 was determined by the p-Nitrophenyl Phosphate (pNPP) colorimetric assay in which pNPP was used substrate and SHP2 activity was reflected by the increase of 450 nm absorbance.

FIGS. 12A-12B illustrate effects of combination of YIV-906 and nivolumab on NFAT mediated transcriptional activity of PD1 overexpressed Jurkat cells incubated with wild type Raji or PD-L1 overexpressed Raji cells with 10 ng/ml (FIG. 12A) or 30 ng/ml (FIG. 12B) staphylococcal superantigen E (SEE). Jurkat-PD1 cells was treated with biosimilar nivolumab 18 ug/ml for 24 h and then YIV-906 160 ug/ml water extract and wild type Raji cells or Raji-PD-L1 cells were added to Jurkat PD1 cells for another 24 h before luciferase activity was measured.

FIGS. 13A-13F illustrate effects of YIV-906, S and S compounds on NFAT mediated transcriptional activity of Jurkat cells triggered by the interaction of chimeric antigen receptor (CAR-CD19-CD3z) on Jurkat cells and CD19 on Raji cells. Effects of YIV-906 (FIG. 13A), S (FIG. 13A) and S compounds (FIG. 13B) on NFAT mediated transcriptional activity of Jurkat-PD1 cells carrying chimeric antigen receptor (CAR-CD19-CD3z) are shown. Effects of YIV-906 (FIG. 13C), S (FIG. 13C) and S compounds (FIG. 13D) on NFAT mediated transcriptional activity of Jurkat-PD1 cells carrying chimeric antigen receptor (CAR-CD19-CD3z) and incubated with wild type Raji cells are also shown. Effect of YIV-906 (FIG. 13E), S (FIG. 13E) and S compounds (FIG. 13F) on NFAT mediated transcriptional activity of Jurkat-PD1 cells carrying chimeric antigen receptor (CAR-CD19-CD3z) and incubated with Raji-PD-L1 cells. YIV-906, S up to 320 ug/ml water extract and S compounds 40 uM were added to Jurkat-PD1-CAR-CD19 cells, which were stably transfected with NFAT luciferase reporter, together with wild type Raji cells or Raji-PD-L1 cells for 24 h before luciferase activity was measured.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions

As used herein, each of the following terms has the meaning associated with it in this section.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, exemplary methods and materials are described.

Generally, the nomenclature used herein and the laboratory procedures in pharmacology, natural product chemistry, and organic chemistry are those well-known and commonly employed in the art.

As used herein, the articles “a” and “an” refer to one or to more than one (i.e., to at least one) of the grammatical object of the article. By way of example, “an element” means one element or more than one element.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “extract” refers to a concentrated preparation or solution of a compound or drug derived from a naturally occurring source, such as an herb or other plant material. Extracts may be prepared by a number of processes, including steeping an herb in solution, or drying and grinding an herb into a powder and dissolving the powder in a solution. An extract may be further concentrated by removing a portion of the solvent after dissolving an amount of the desired compound in the solution. An extract may also be strained or centrifuged to remove any solid material from the solution.

The phrase “inhibit,” as used herein, means to reduce a molecule, a reaction, an interaction, a gene and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein or a gene's stability, expression, function and activity, e.g., antagonists.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the term “YIV-906” refers to an herbal composition comprising Glycyrrhiza uralensis Fisch (G), Paeonia lactiflora Pall (P), Scutellaria baicalensis Georgi(S), and Ziziphus jujuba Mill (Z). YIV-906 can refer to, for example, to a specific composition comprising S, G, P and Z in a 3:2: 2:2 ratio prepared under standard operational procedures, including, in some embodiments, hot water extraction of S, P, G, and Z.

As used herein, the term “prevent,” “prevention,” or “preventing” refers to any method to partially or completely prevent, delay, or slow the onset of one or more symptoms or features of a disease, disorder, and/or condition, for example, cancer. Prevention is causing the clinical symptoms of the disease state not to develop, i.e., inhibiting the onset of disease, in a subject that may be exposed to or predisposed to the disease state, but does not yet experience, or display symptoms of the disease state. Prevention may be administered to a subject who does not exhibit signs of a disease, disorder, and/or condition. In some embodiments, slowing the onset of one or more symptoms or features of a disease or disorder means that, if a recurrence of the disease or disorder or one or more symptoms of the disease or disorder occurs, then the disease or disorder or one or more symptoms of the disease or disorder recur at least about 5%, 10%, 20%, 30%, 40%, 50%, 60%, 70%, 80%, 90%, 95%, or 99% slower than the disease or disorder or one or more symptoms of the disease or disorder would recur in the absence of administering YIV-906.

As used herein, the term “subject,” “patient” or “individual” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, the term “therapeutically effective amount” is an amount of a compound of the disclosure, that when administered to a patient, treats, minimizes, and/or ameliorates a symptom of the disease or disorder. The amount of a compound of the disclosure that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

As used herein, the term “treatment” or “treating” is defined as the application or administration of a therapeutic agent, i.e., a compound useful within the disclosure (alone or in combination with another pharmaceutical agent), to a subject, or application or administration of a therapeutic agent to an isolated tissue or cell line from a subject (e.g., for diagnosis or ex vivo applications), who has cancer, a symptom of cancer or the potential to develop cancer, with the purpose to cure, heal, alleviate, relieve, alter, remedy, ameliorate, improve or affect cancer, the symptoms of cancer or the potential to develop cancer. Such treatments may be specifically tailored or modified, based on knowledge obtained from the field of pharmacogenomics.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

Additionally, throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The following abbreviations are used herein:
CGMP current Good Manufacturing Practice
CAR Chimeric antigen receptor
NFAT Nuclear factor of activated T-cells
TCR T Cell Receptor
SHP2 SH2-containing phosphatase 2
SEE staphylococcal superantigen E
G Glycyrrhiza uralensis Fisch, also known as Chinese liquorice
P Paeonia lactiflora Pall, also known as Chinese peony
S Scutellaria baicalensis Georgi, also known as Baikal skullcap or scute
Z Ziziphus jujube Mill, also known as red date or Chinese date.

YIV-906 is inspired by a traditional Chinese herbal formulation and is composed of four herbs: Glycyrrhiza uralensis Fisch (G), Paeonia lactiflora Pall (P), Scutellaria baicalensis Georgi (S), and Ziziphus jujuba Mill (Z). Historically, YIV-906 was used to treat gastrointestinal symptoms including diarrhea, nausea, and vomiting. Here, a Jurkat cells-staphylococcal superantigen E (SEE)-Raji cells cell culture model is used to demonstrate that YIV-906 could promote T cell activation with up-regulation of CD69 by modulating nuclear factor of activated T-cells (NFAT) activity. Without wishing to be bound by any theory, this might be due to that YIV-906 could modulate SHP2 activity leading to increased protein phosphorylation of T cell receptor signaling cascades including, Lck, Fyn, Zap70, LAT and Pyk2. G, S and their compounds showed inhibitory effect on SHP2. P, S and their compounds could induce protein phosphorylation of T cell receptor signaling cascades. S and flavonoids of S of YIV-906 are found to play key roles of modulating NFAT activity. Importantly, YIV-906 could enhance Anti-PD1 action to rescue the depressed NFAT activity of Jurkat cells-PD1 cells when incubated with Raji-PD-L1 cells. In addition, YIV-906 could also enhance NFAT activity triggered by the interaction of chimeric antigen receptor (CAR-CD19-CD3z) on Jurkat cells and CD19 on Raji cells. It is demonstrated herein that YIV-906 and some of its components could modulate T cell activation to enhance the effectiveness of immune checkpoint blockade therapy, CAR T-cell therapy, and vaccines.

### Compositions

In one aspect, the present invention provides an herbal composition for modulating NFAT activity in T cells of a subject in need thereof.

In certain embodiments, the composition comprises an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;

In certain embodiments, the composition comprises an herbal extract of S, a fraction thereof or any active chemical present in the herbal extract or fraction thereof. In certain embodiments, the composition comprises an herbal extract comprising an extract of S, a fraction thereof or any active chemical present in the herbal extract or fraction thereof.

In certain embodiments, the composition comprises an herbal extract of G, a fraction thereof or any active chemical present in the herbal extract or fraction thereof. In certain embodiments, the composition comprises an herbal extract comprising an extract of G, a fraction thereof or any active chemical present in the herbal extract or fraction thereof.

In certain embodiments, the composition comprises an herbal extract of P, a fraction thereof or any active chemical present in the herbal extract or fraction thereof. In certain embodiments, the composition comprises an herbal extract comprising an extract of P, a fraction thereof or any active chemical present in the herbal extract or fraction thereof.

In certain embodiments, the composition comprises an herbal extract of Z, a fraction thereof or any active chemical present in the herbal extract or fraction thereof. In certain embodiments, the composition comprises an herbal extract comprising an extract of Z, a fraction thereof or any active chemical present in the herbal extract or fraction thereof.

In certain embodiments, the composition further comprises one or more pharmaceutically acceptable carriers.

In certain embodiments, the composition is formulated in the form of a pill, a tablet, a capsule, a soup, a tea, a concentrate, a dragee, a liquid, a drop, or a gelcap for oral administration.

In certain embodiments, the composition is used for facilitating immune checkpoint blockade therapy for cancer treatment, facilitating CAR T cell therapy for cancer treatment, enhancing the effectiveness of vaccination and/or as cancer chemoprevention agents.

### Methods

In another aspect, the invention provides a method for enhancing NFAT activity and/or restoring the suppressed NFAT activity in T cells of a subject in need thereof. Additionally, the invention also includes a method of enhancing efficacy of Chimeric Antigen Receptor T-cell (CAR T) immunotherapy in a subject; a method of enhancing efficacy of immune checkpoint blockade therapy in a subject; a method of enhancing effectiveness of a vaccine in a subject; and a method for cancer chemoprevention.

In certain embodiments, the method comprises administering to the subjects an effective amount of an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof.

In certain embodiments, the method comprises administering to the subjects an effective amount of an herbal extract of S, a fraction thereof or any active chemical present in the herbal extract or the fraction thereof. In certain embodiments, the method comprises administering to the subjects an effective amount of an herbal extract comprising an extract of S, a fraction thereof or any active chemical present in the herbal extract or the fraction thereof. In certain embodiment, the active chemical in the herbal extract of S or the fraction thereof is at least one selected from the group consisting of oroxylin A 7-O-beta-D-glucuronide, baicalein, oroxylin A, baicalin, isomer of baicalin, chrysin 6-C-arabinoside 8-C-glucoside, acteoside, viscidulin I 2′-O-glucoside and chrysin or any combination thereof.

In certain embodiments, the method comprises administering to the subjects an effective amount of an herbal extract of G, a fraction thereof or any active chemical present in the herbal extract or the fraction thereof. In certain embodiments, the method comprises administering to the subjects an effective amount of an herbal extract comprising an extract of G, a fraction thereof or any active chemical present in the herbal extract or the fraction thereof. In certain embodiments, the active chemical in the herbal extract of G or the fraction thereof is at least one selected from the group consisting of liquiritigenin, isoliquiritigenin, liquiritin, isoliquiritin, licochalcone A, and glycycoumarin.

In certain embodiments, the method comprises administering to the subjects an effective amount of an herbal extract of P, a fraction thereof or any active chemical present in the herbal extract or the fraction thereof. In certain embodiments, the method comprises administering to the subjects an effective amount of an herbal extract comprising an extract of P, a fraction thereof or any active chemical present in the herbal extract or the fraction thereof. In certain embodiment, the active chemical in the herbal extract of P or the fraction thereof is at least one selected from the group consisting of gallic acid, 4-o-methyl paeoniflorin, 4-o-methyl paeoniflorin isomer, 6′-O-galloyl paeoniflorin, albiflorin, paeoniflorin or any combination thereof.

In certain embodiments, the method induces protein phosphorylation of TCR downstream cascades and inhibits SHP2 activity.

In certain embodiments, the methods described herein are used for increasing efficacy of anti-cancer agents/immunotherapeutic agents including Ipilimumab, Pembrolizumab, Nivolumab, Durvalumab, Avelumab, Dostarlimab-gxly, Cemiplimab-rwlc, Toripalimab, Camrelizumab, Tislelizumab, Penpulimab, Zimberelimab, Sintilimab, Atezolizumab, and Chimeric Antigen Receptor T-cells (CAR T cells) or any combination thereof. In certain embodiments, the methods of the invention are used for treating cancers such as, for example, melanoma, Merkle cell carcinoma (MCC), basal cell carcinoma (BCC), squamous cell carcinomas including, for example, head and neck squamous cell carcinoma (HNSCC), esophageal squamous cell carcinoma (ESCC), and cutaneous squamous cell carcinoma (CSCC), lymphomas including, for example, Hodgkin's lymphoma, classic Hodgkin lymphoma (cHL) and large B cell lymphoma, lung cancers including, for example, non-small cell lung cancer (NSCLC), small cell lung cancer (SCLC), and extensive-stage small cell lung cancer (ES-SCLC), liver cancers including hepatocellular carcinoma (HCC), pancreatic cancer, renal cell carcinoma (RCC), colon cancer, colorectal cancer (CRC), mesothelioma, gastric cancer, gastroesophageal junction cancer, esophageal carcinomas including esophageal adenocarcinoma, cervical cancer, endometrial carcinomas including mismatch repair deficient (dMMR) recurrent or advanced endometrial cancer, bladder cancers including urothelial carcinoma, breast cancers including triple-negative breast cancer (TNBC), Mismatch repair (MMR) deficient cancers, microsatellite instability (MSI)-high cancer, and tumor mutational burden (TMB)-high cancers.

In certain embodiments, the method comprises administering the herbal extracts to the subject orally in form of a pill, a tablet, a capsule, a soup, a tea, a concentrate, a dragee, a liquid, a drop, and a gelcap.

In certain embodiment, the herbal composition further comprises a pharmaceutically acceptable carrier.

In certain embodiments, the therapeutically effective amount of the herbal composition is about 20 mg/day to about 2 g/day.

In certain embodiments, the therapeutically effective amount of the herbal composition is about 1,600 mg/day.

In certain embodiments, the herbal composition is administered at a time selected from prior to, simultaneously with, and after administration of the one or more immunotherapeutic agent or a vaccine.

In certain embodiments, the subject is a mammal. In certain embodiments, the subject is a human subject.

### Administration/Dosage/Formulations

The regimen of administration may affect what constitutes an effective amount. The therapeutic formulations may be administered to the subject either prior to or after the onset of disease or disorder contemplated in the disclosure. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat a disease or disorder contemplated in the disclosure. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the disclosure. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 1,000 mg/kg of body weight/per day. The pharmaceutical compositions useful for practicing the disclosure may be administered to deliver a dose of from 1 ng/kg/day and 100 mg/kg/day. One of ordinary skill in the art would be able to study the relevant factors and make the determination regarding the effective amount of the therapeutic compound without undue experimentation.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the disclosure.

In certain embodiments, the compositions of the disclosure are formulated using one or more pharmaceutically acceptable excipients or carriers. In other embodiments, the pharmaceutical compositions of the disclosure comprise a therapeutically effective amount of a compound of the disclosure and a pharmaceutically acceptable carrier. In yet other embodiments, the compound of the disclosure is the only biologically active agent (i.e., capable of treating cancer) in the composition. In yet other embodiments, the compound of the disclosure is the only biologically active agent (i.e., capable of treating cancer) in therapeutically effective amounts in the composition.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds and/or compositions of the disclosure for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 400 mg to about 6,000 mg, about 500 mg to about 5,000 mg, about 750 mg to about 4,000 mg, about 1,000 mg to about 3,000 mg, about 1,000 mg to about 2,500 mg, about 20 mg to about 2,000 mg and any and all whole or partial increments therebetween. In certain embodiments, the dose of the compounds and/or compositions of the disclosure is about 800 mg.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated in the disclosure.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the disclosure include oral nasal, rectal, intravaginal, parenteral, buccal, sublingual, or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-peritoneal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are soups, teas, concentrates, tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present disclosure also includes a multi-layer tablet comprising a layer providing for the delayed release of one or more compounds of the disclosure, and a further layer providing for the immediate release of a medication for treatment of a disease or disorder contemplated in the disclosure. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intra-peritoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1, 3-butanediol, for example. Other acceptable diluents and solvents include, but are not limited to, Ringer's solution, isotonic sodium chloride solution, and fixed oils such as synthetic mono-or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds useful within the methods of the disclosure may be administered in the form of microparticles, for example by injection, or in the form of wafers or discs by implantation.

In one embodiment of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present disclosure depends on the age and weight of the patient, the current medical condition of the patient and the progression of a disease or disorder contemplated in the disclosure. The skilled artisan is able to determine appropriate dosages depending on these and other factors.

A suitable dose of a compound, composition, or extract of the present disclosure can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

In various embodiments, the amount or dose of the herbal composition administered can be from about 0.5 mg/kg to about 5000 mg/kg, about 1 mg/kg to about 2500 mg/kg, about 5 mg/kg to about 1000 mg/kg, or about 10 mg/kg to about 1000 mg/kg. In various embodiments, the amount or dose of the YIV-906 herbal extract administered can be about 0.01, 0.5, 1, 2, 3, 4, 5,6, 7, 8, 9, 10, 20, 30, 40, 50, 60, 70, 80, 90, 100, 120, 140, 160, 180, 200, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, 1800, 1820, 1840, 1860, 1880, 1900, 1920, 1940, 1960, 1980, 2000, 2500, 3000, 3500, 4000, 4500, or about 5000 mg/kg. These amounts of herbal extract can be administered using any of the dosing regimens described herein.

In various embodiments, the amount or dose of any immune checkpoint inhibitor or immunotherapeutic agent described herein can be from about 0.01 mg/kg to about 50 mg/kg, about 0.05 mg/kg to about 30 mg/kg, or about 1 mg/kg to about 20 mg/kg. In various embodiments, the amount or dose of any immune checkpoint inhibitor or immunotherapeutic agent described herein can be 0.01, 0.05, 0.1, 0.2, 0.3, 0.4, 0.5, 0.6, 0.7, 0.8, 0.9, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, 15, 15.2, 15.4, 15.6, 15.8, 16, 16.2, 16.4, 16.6, 16.8, 17, 17.2, 17.4, 17.6, 17.8, 18, 18.2, 18.4, 18.6, 18.8, 19, 19.2, 19.4, 19.6, 19.8, or about 20 mg/kg. In some embodiments, the maximum administered daily amount or dose of any immune checkpoint inhibitor or immunotherapeutic agent described herein can be about 10, 20, 30, 40, 50, 60, 70, 80, 90, 100, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, 1800, 1820, 1840, 1860, 1880, 1900, 1920, 1940, 1960, 1980, or about 2000 mg.

In some embodiments, the herbal composition and a single immunotherapeutic agent are the only therapeutically active agents in a pharmaceutical composition.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

The disclosure is further described in detail by reference to the following experimental examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

### Methods and Materials

### Preparation of Herbal Extracts

PHY906 is comprised of a traditional hot water extract of four herbs, Scutelleria baicalensis Georgi (S), Paeonia lactiflora Pall. (P), Glycyrrhiza uralensis Fisch. (G), and Ziziphus jujuba Mill (Z), in the ratio of 3:2:2:2, respectively, and prepared under standard operational procedure. This extract comprises a complex mixture of multiple phytochemicals with multiple biological and pharmacological properties. At this time, it is not possible to identify the subset of relevant biologically active phytochemicals from the entire mixture. For this reason, a high-level chemical and biological metrics to characterize the PHY906 product was used.

The raw ingredients of PHY906 are pre-selected to meet rigid specifications set by PhytoCeutica for acceptance by the herbal manufacturer, Sun Ten Pharmaceuticals in Taiwan. The PHY906 extract is comprised of greater than 75% low molecular weight phytochemical compounds less than 1000 amu, 10% macromolecular components including protein, nucleic acid, complex carbohydrates, and 5% water. In addition, 10% by weight of excipient insoluble cellulose is added during the spray dry step in manufacturing. Heavy metals (Pb, Hg, Cd, As) are all less than 0.5 ppm, with mercury and cadmium less than 0.03 ppm, as detected by atomic absorption measurements. Pesticides levels (BHCs, DDTs, PCNB) are less than 0.2 ppm by LC-MS or GC-MS. Total bacteria counts are 260 cfu/g while E. coli and Salmonella species are not detected. Over 90% by weight of PHY906, excluding water content (5%) and insoluble starch excipient (10%), can be re-extracted. The final PHY906 liquid extract (100 mg/ml) is stable for 18 hours at room temperature and the properly stored bulk dry extract (vacuum packed, light tight and 4° C.) appears to be stable for more than three years.

### LC-MS Methods

### LC-MS Analysis for Chemical Profiles of the Metabolites of PHY906

The LC-MS analysis was performed on an Agilent 1200 series HPLC coupled with AB SCIEX 4000 QTRAP mass spectrometer. The separation was conducted on an Alltima™ HP HPLC Column (5 mm, 4.6×250 mm). The mobile phase was acetonitrile (A) and water with 0.1% formic acid (B) with gradient elution: 0 min, 5% A; 10 min, 20% A; 20 min, 25% A; 40 min, 30% A; 45 min, 35% A; 55 min, 45% A; 60 min, 70% A; 62 min, 90% A; 67 min, 90% A; 68 min, 5% A; and 75 min, 5% A. The flow rate was 1.0 mL/min, and the column temperature was set at 30° C. ESI negative mode mass spectrometry of scan rate 4000 amu/s was performed with the following ionization parameters: CAD: High; TEM: 550.00° C.; GS1: 55.00; GS2: 50.00; ihe: ON; IS: −4250.00; DP: −40.00; CES 0.00; CE: −5.00. The mass range for detection was 120-800 amu. Using a custom program integrated with MZmine software, the peaks were compared, and a clustering analysis was created

### Cell Lines

RPMI1640+5% FBS+50 ug/ml Kanamycin at 37° C. with 5% CO₂ incubation was the condition for cell culture and experiment. Jurkat (T lymphoblast) cells were transfected with pcDNA-PD1-Dyk DNA and then selected by G418 200 ug/ml. Raji (B-lymphocyte) cells were transfected with pcDNA-PD-L1-Dyk DNA and then selected by G418 600 ug/ml. Anti-PD1-PE (biolegend, #A17188B) or Anti-PD-L1 (CD274)-APC (invitrogen, #17-5983-42) were used to confirm the expression of PD1 or PD-L1 on Jurkat cells or Raji cells using laser flow cytometer (BD, Accuri 6 plus). Jurkat-PD1 cells were transfected with NFAT luciferase reporter DNA in which four repeated NFAT response element: ggaggaaaaactgtttcatacagaaggcgt (SEQ ID NO: 1) oligo had been inserted into pGL4 vector (promega) and then selected with 0.1 ug/ml puromycin. Jurkat-PD1 NFAT-luc TCRαβ knockout cells were generated by using CRISPR/Cas9 technology. Targeting DNA sequence for TCRa and TCRb are CTTCAAGAGCAACAGTGCTG (SEQ ID NO: 2) and AGGTCGCTGTGTTTGAGCCA (SEQ ID NO: 3) respectively. Anti-TCRαβ (Biolegend) was used to confirm knock out of TCRαβ. CAR-CD19-CD3zeta vector was transfected to Jurkat-PD1 NFAT-luc and GFP positive cells were sorted out using flow cytometer. Jurka-PD1 NFAT-luc CAR-CD19-CD3zeta clones were selected when NFAT luciferase activity was increased by coculturing with wild Raji cells.

### Stimulation for NFAT of Jurkat Cells

75 ul of Jurkat-PD1 cells at 10⁶ with or without wild type Raji cells or Raji-PD-L1 cells at 2×10² were seeded into round bottom 96-well plate. In some experiments, In VivoSIM anti-human PD-1 (Nivolumab Biosimilar) (BioXcell, #SIM0003) 18 ug/ml was added to the cells for 2 h or 24 h before drug and SEE treatment. 25 ul of control medium or 25 ul of 5× concentration of YIV-906 or other drugs were added to the well. 25 ul of SEE (Toxin Technology, #ET404) (5×10 ng/ml) was then added to the well. After incubating overnight at at 37° C. with 5% CO₂, cells were lysed using luciferase lysis buffer. Luciferase buffer with luciferin was added to generate luminescence. Luminescence was recorded using a luminescence microplate reader.

### Flow Cytometry Analysis

Jurkat-PD1 cells 2×10⁵/ml with or without wild type Raji cells or Raji-PD-L1 cells 4×10⁵/ml in 24 well plate were treated with YIV-906 with or without SEE (10 ng/ml and 30 ng/ml) for 48 h. Anti-CD69-FITC (biolegend, #310904) and Anti-PD1-PE (biolegend, #A17188B) in PBS+1% BSA were used to stained the cells. Anti-PD1-PE was used to gate Jurkat-PD1 cells from the mixed cells. The expression of CD69 in FITC channel was present as median fluorescence or % of total Jurkat cells population using laser flow cytometer (BD, Accuri 6 plus).

### Western Blot Analysis

Jurkat-PD1 cells 10⁶/ml were placed in 24 well plate treated with YIV-906 or its components herbs for 45 minutes. After spinning down the cells at 1000 g centrifugation for 10 min, red blood cells were lysed with 1 ml BD pharm lyse on ice. Cells were collected at 1000 g centrifugation for 10 min. 2×10⁶ cells were used for each staining sample. The cells were prepared with 2X protein loading buffer (Tris pH 6.8 1M, SDS 1%, glycerol, B-mercaptoethanol, bromophenol blue, distilled water). The samples were then heated to 95° C. for 5 minutes to denature the proteins prior to western blot. SDS polyacrylamide gel electrophoresis (10% Mini-Protean TGX™ Precast Protein Gels, BioRad) was used to separate proteins according to their electrophoretic mobility. The protein extractions were deposited in the wells of the gel, 10 μL per well. The migration was performed in a 1X running buffer (Tris/Glycine/SDS) at 180 V for 50 minutes. The transfer of the proteins was performed on a PVDF membrane in the transfer buffer (Tris-CAPS AX, methanol, SDS 10%, distilled water) at 75 V for 1 hour. The non-specific binding sites on the PVDF membrane were blocked with a blocking solution (3% milk powder and 1X TBS-T) for 30 minutes. Then, the PVDF membrane was incubated with the primary antibody against the proteins of interest overnight at 4° C. The information of primary antibodies is as follows: P-Lck-Y394 (Biolegend, #933101), P-Zap70-Y319 (Cell signaling, #2717), P-LAT-Y191 (Cell signaling, #3584) P-SRC (Fyn) Y416 (Cell, signaling, #6943), P-Pyk2-Y402 (Cell signaling, 3291), GAPDH (Cell Signaling, #5174). The membrane was washed with TBS-T 1X and incubated with secondary antibody with horseradish peroxidase-conjugated anti-rabbit IgG 1:5000 (ThermoFisher Scientific, #A27036) against the immunoglobulin corresponding to the primary antibody for 1 hour at room temperature. The membrane was washed with TBS-T 1X. The protein bands were detected using chemiluminescence (Super Signal West Dura, Thermo Scientific Cat #PI34076) and the images were revealed by an X-Ray Film Processor (Fuji Super RX-N). The densitometry scanning was performed with the Epson V600 scanner. Image-J was used to quantify the total intensity of immunoreactive bands. GAPDH was used as an internal control for normalization.

### Example 1: YIV-906 Could Modulate Nuclear Factor of Activated T-Cells (NFAT) Activity of T Cells

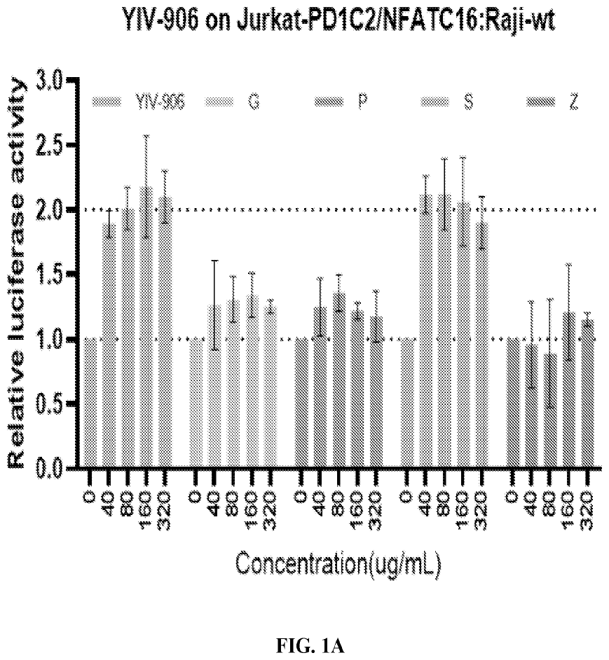

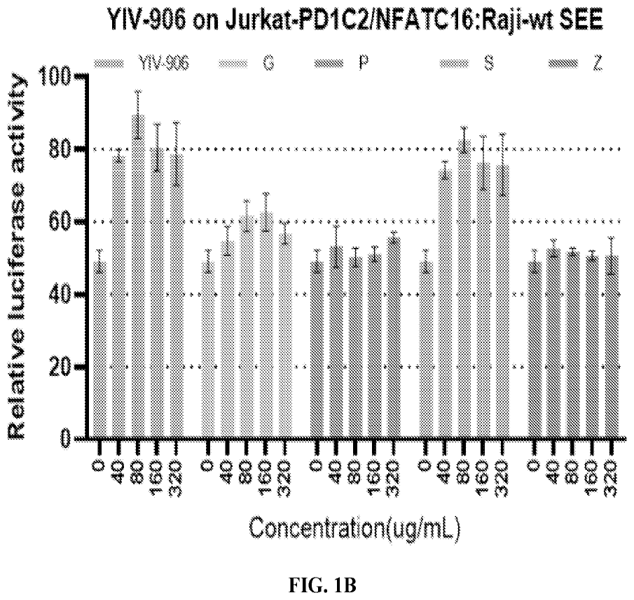

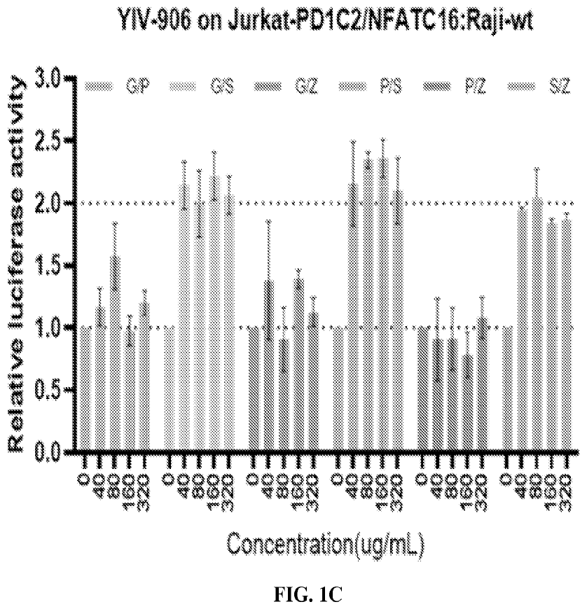

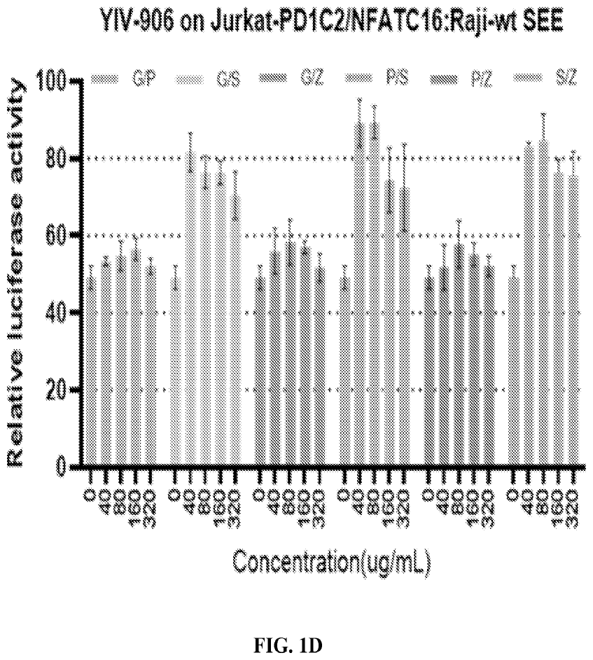

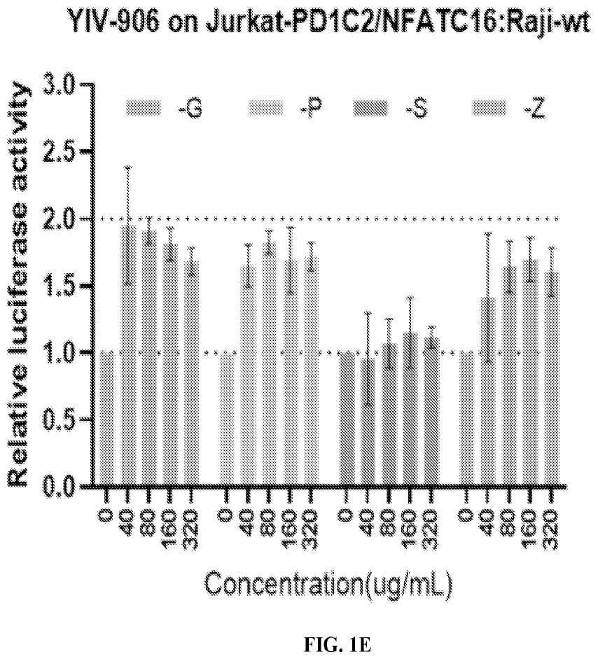

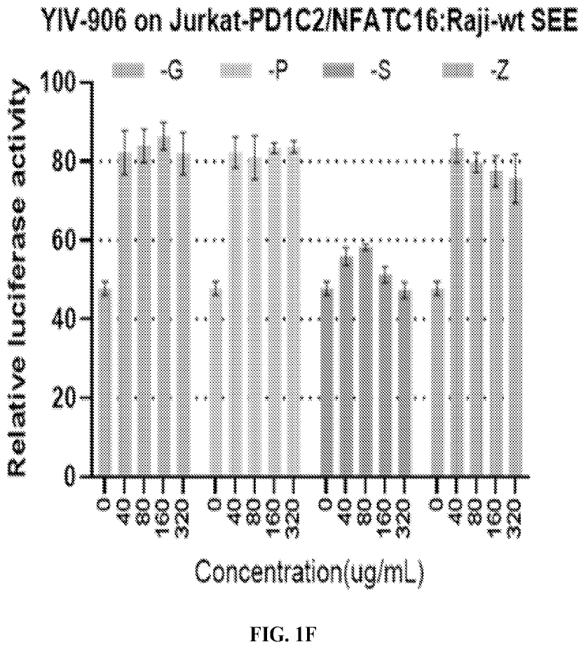

Jurkat cells-staphylococcal superantigen E (SEE)-Raji cells cell culture model was established for examining the impacts of YIV-906 on T cell activation. In this cell culture model, Jurkat cells (T cell) were stably transfected with nuclear factor of activated T-cells (NFAT) luciferase reporter DNA and PD1 DNA. For stimulation, Jurkat cells-PD1 cells were co-cultured with wild type Raji cells in 1:2 ratio and SEE 1 ng/ml to 10 ng/ml in absence or presence of YIV-906 or its components that were added to the mixed cell for overnight. NFAT activity was reflected by luciferase activity as the measurement of chemiluminescence. From the above cell culture model, it was concluded that YIV-906 was able to modulate NFAT in either absence (FIG. 1A) or presence (FIG. 1B) of SEE. YIV-906, at the range 80 ug/ml to 320 ug/ml, could promote NFAT activity by one-fold. When YIV-906 was compared to its component herbs (at their equivalent concentration): Glycyrrhiza uralensis Fisch (G), Paeonia lactiflora Pall (P), Scutellaria baicalensis Georgi (S), and Ziziphus jujuba Mill (Z) for NFAT mediated transcriptional activity of Jurkat cells, S showed very similar dose response to YIV-906 in both absence and presence of SEE (FIGS. 1A and 1B). G and P could modulate no more than 20% NFAT activity in absence or presence of SEE (FIGS. 1A and 1B). Z did not have impact of NFAT activity (FIGS. 1A and 1B). When comparing two herbs' combinations or three herbs' combinations (FIGS. 1C to 1F), only the combinations with S showed about 1-fold enhancement of NFAT activity in absence or presence of SEE. Thus, is demonstrated herein that YIV-906 could modulate nuclear factor of activated T-cells (NFAT) activity of Jurkat cells and that S play an important role in modulating NFAT while G and P might play minor roles in modulating NFAT.

### Example 2: YIV-906 Could Modulate the Depressed Nuclear Factor of Activated T-Cells (NFAT) Activity of T Cells Due to Interaction of PD1 and PD-L1

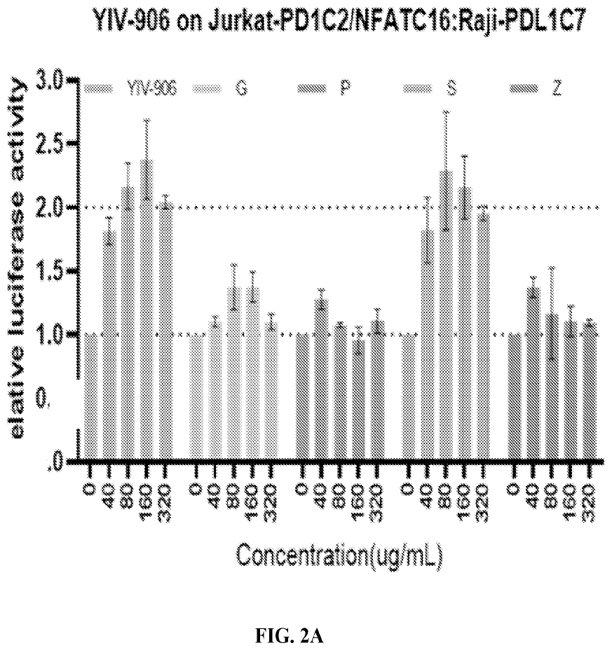

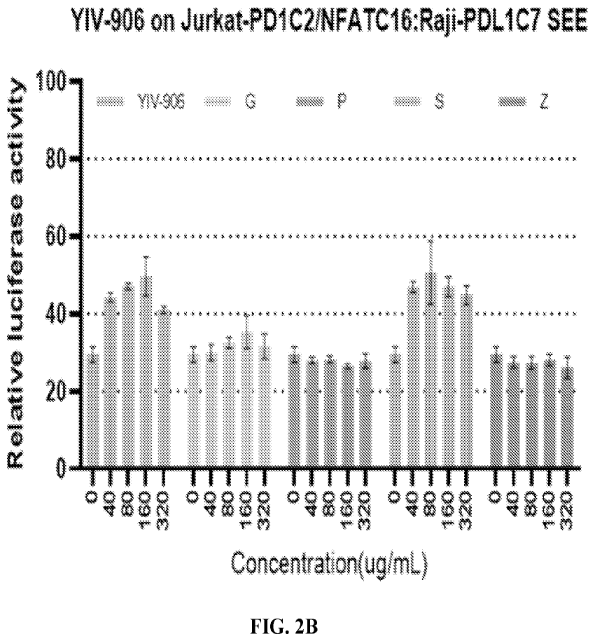

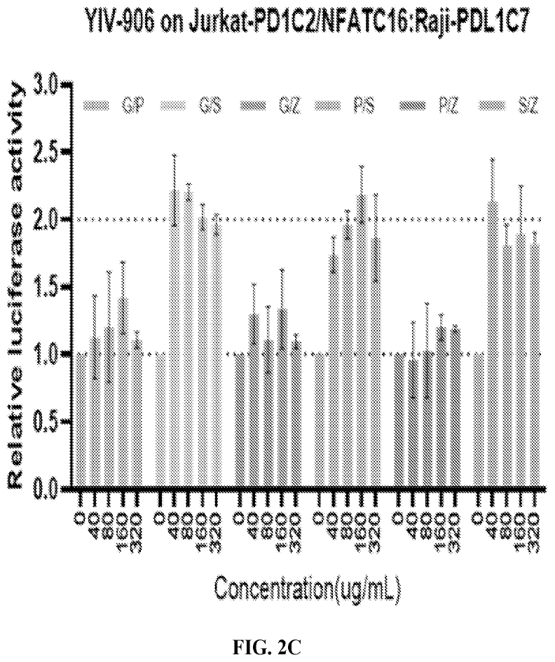

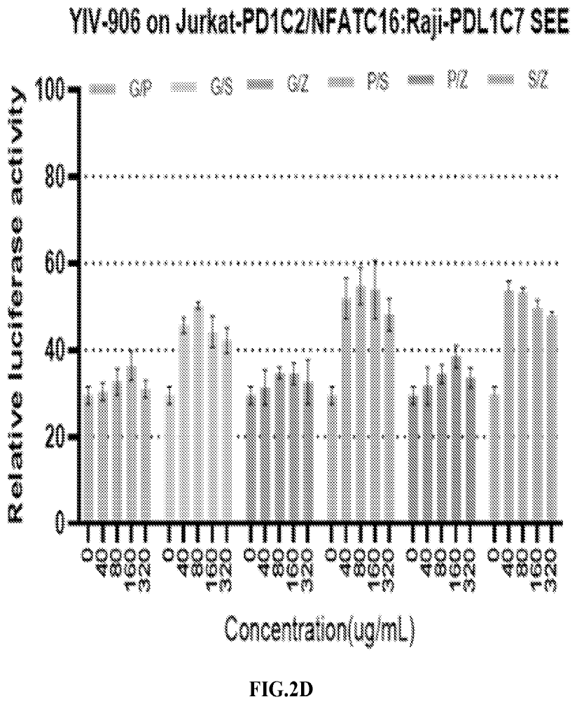

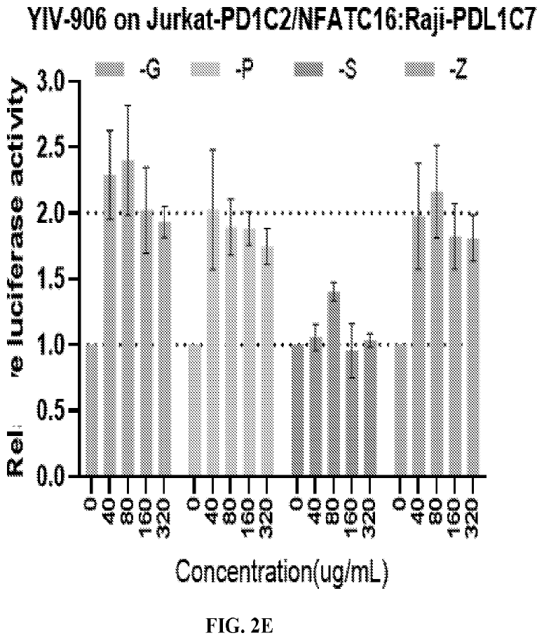

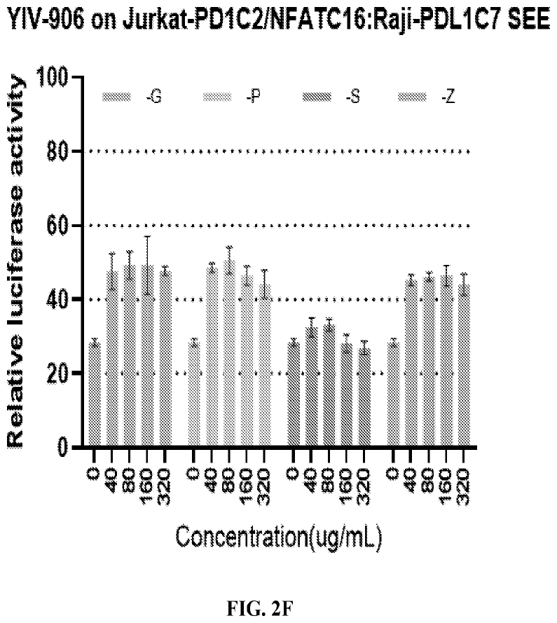

For studying the impact of PD1-PD-L1 on NFAT activity during the T cell activation, Jurkat cells-PD1 cells were co-cultured with PD-L1 over-expressed Raji cells in 1:2 ratio for 2 hours and then SEE 1 ng/ml to 10 ng/ml in absence or presence of YIV-906 or its components that were added to the mixed cell for overnight. YIV-906, S and combinations with S had similar impact on the basal NFAT activity of Jurkat-PD1 cells incubated either with wild type Raji (FIGS. 1A, 1C-1F) or PD-L1 over-expressed Raji cells (FIGS. 2A, 2C-2F). Interaction of PD1 on Jurkat cells and PD-L1 on Raji cells inhibited NFAT activity of Jurkat cells by 50% (FIG. 1B vs FIG. 2B). YIV-906, S and combinations with S could rescue the depressed NFAT activity of Jurkat-PD1 cells (FIGS. 2A, 2C-2F). G had slight impact on NFAT activity in the above conditions (FIGS. 2A-2F).

### Example 3: YIV-906 Could Induce CD69 Expression which is a T Cell Activation Marker

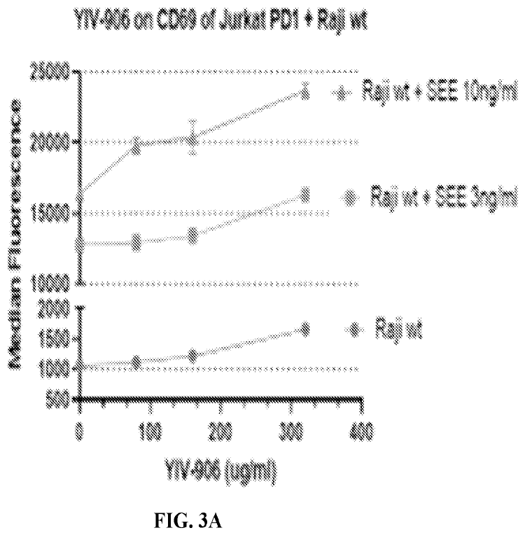

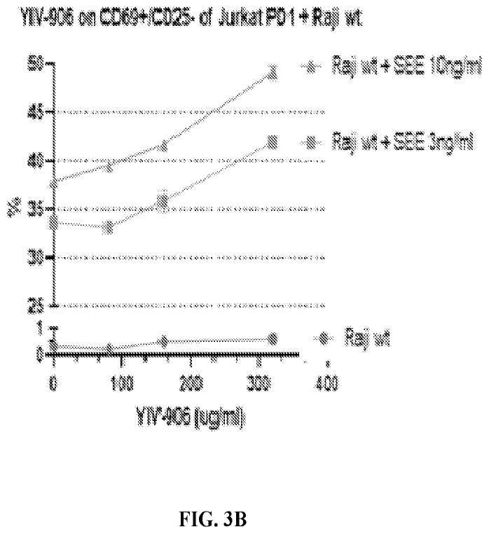

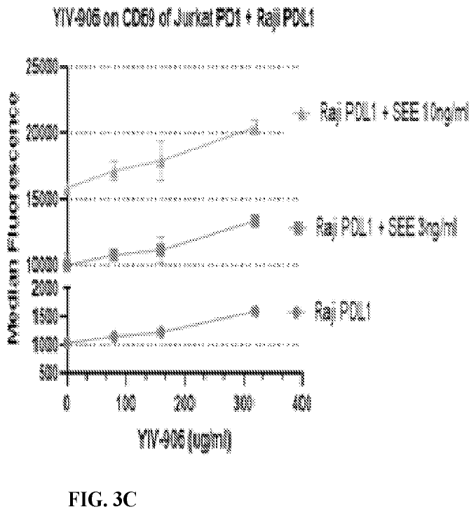

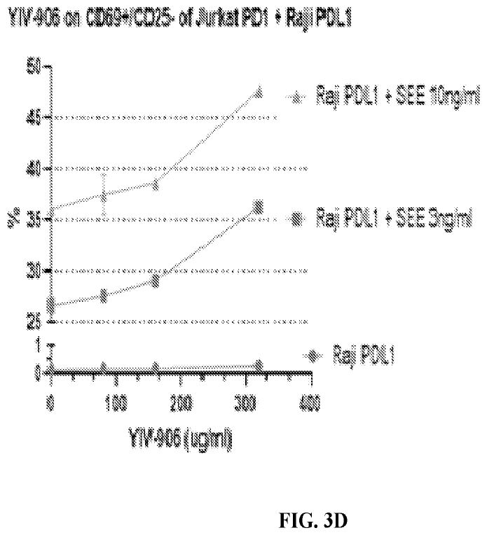

As YIV-906 could modulate NFAT activity, which is an important transcriptional factor for T cell activation, the impact of YIV-906 on T cell activation was further studied by using CD69 as a T cell marker. As shown in FIGS. 3A-3D, YIV-906 can enhance the protein expression of CD69 as indicated by the increase in median fluorescence and % of population with high CD69 staining of Jurkat-PD1 cells when incubated with wild type Raji cells without SEE or with 3 ng or 10 ng/ml SEE (FIGS. 3A and 3B). When Jurkat-PD1 cells were co-cultured with Raji-PD-L1 cells without SEE or with 3ng or 10ng/ml SEE, YIV-906 could increase CD69 expression of Jurkat-PD1 cells. Thus, YIV-906 could modulate NFAT activity of T cells, thereby leading to stronger T cell activation. YIV-906 could also help to restore T cell activation even under the PD1 and PD-L1 interaction.

### Example 4: Pure Compounds of YIV-906 Could Modulate NFAT Activity of T Cells

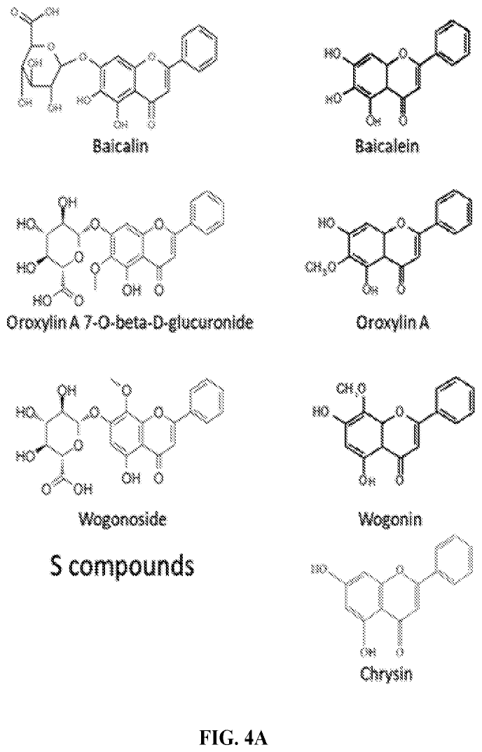

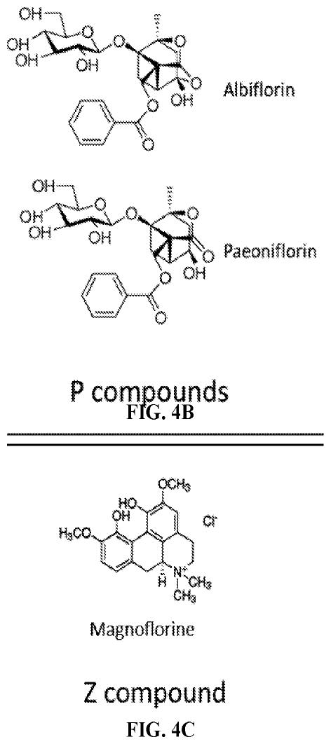

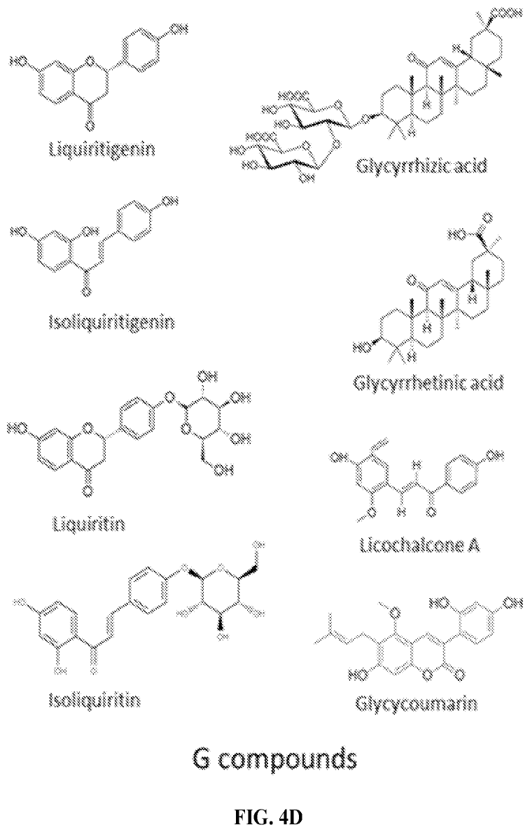

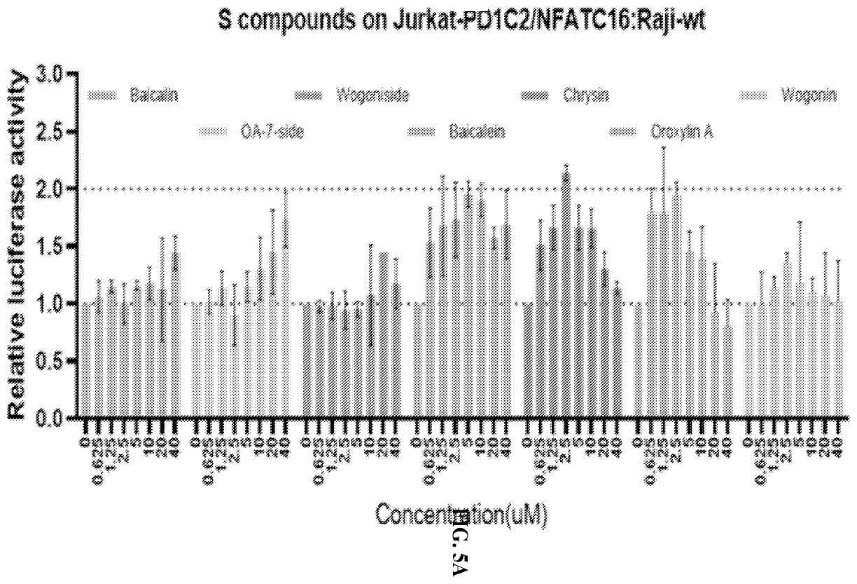

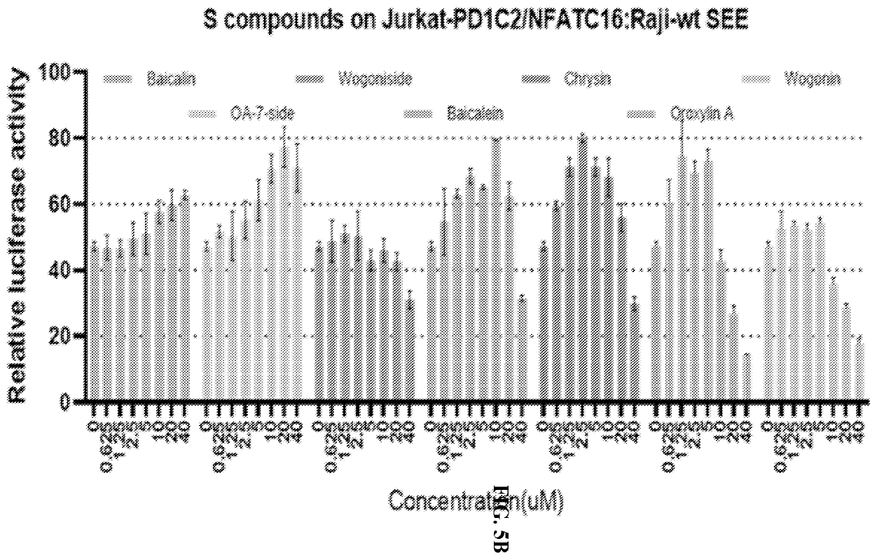

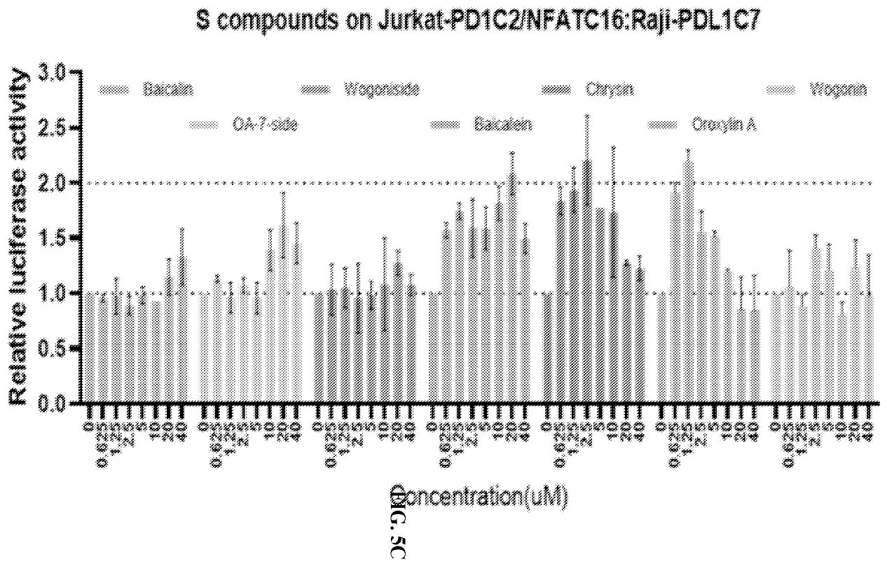

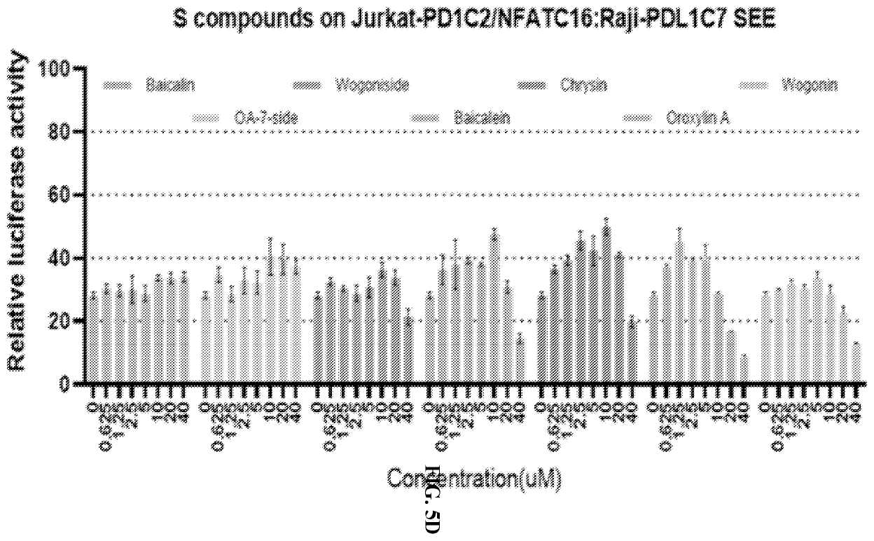

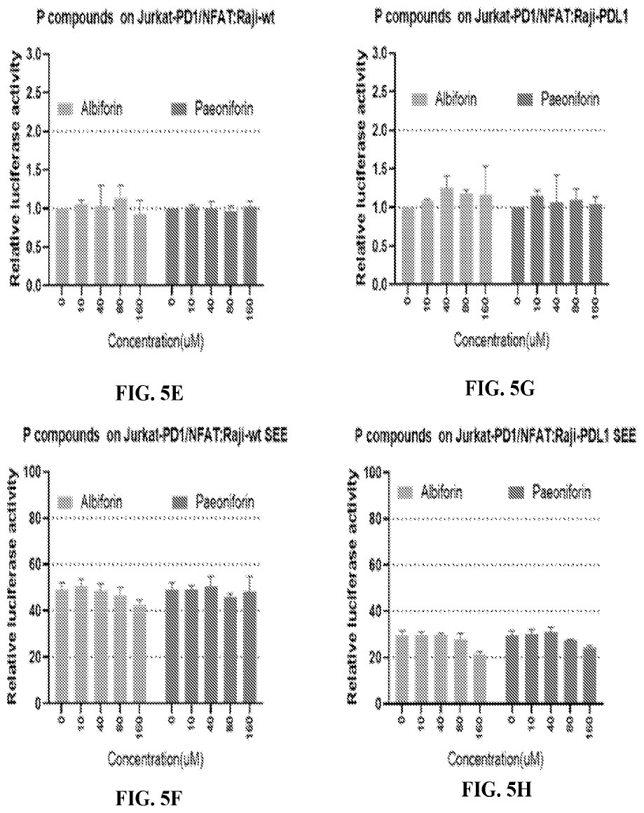

Pure compounds existing in YIV-906 component herbs were selected for examining their impacts on NFAT of Jurkat cells (FIGS. 4A-4D). Among 7 selected compounds from S, oroxylin A 7-O-beta-D-glucuronide, baicalein, oroxylin A, and chrysin could modulate the basal NFAT activity of Jurkat-PD1 cells incubated either with wild type Raji or PD-L1 over-expressed Raji cells (FIGS. 5A and 5C) however, their optimum doses were different. Higher dose (40uM) required for oroxylin A 7-O-beta-D-glucuronide to achieve 2-fold activity of NFAT while baicalein, oroxylin A, and chrysin just required 10 uM to 2.5 uM to have similar impact on NFAT. In presence of SEE, oroxylin A 7-O-beta-D-glucuronide, baicalein, oroxylin A, and chrysin showed similar dose response as they did on basal NFAT activity but baicalein, oroxylin A, and chrysin could inhibit SEE triggered NFAT activity at 20 to 40 uM (FIG. 5B and 5D).

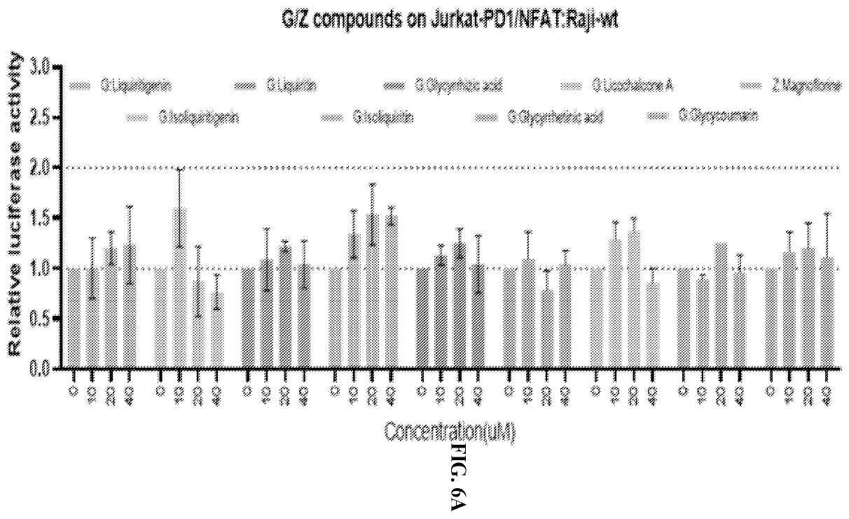

In absence of SEE, liquiritigenin (40 uM), Isoliquiritigenin (10 uM), liquiritin (20 uM), isoliquiritin (40 uM), licochalcone A (10-20 uM) and glycycoumarin (10 uM) from G might have some modulation effect on the basal NFAT activity of Jurkat-PD1 cells either incubated with wild type Raji or PD-L1 over-expressed Raji cells (FIG. 6A and 6C). In presence of SEE, liquiritigenin (40 uM), isoliquiritin (40 uM), licochalcone A (10 uM) and glycycoumarin (20 uM) from G could modulate NFAT activity up to about 30-50% of Jurkat-PD1 cells incubated either with wild type Raji or PD-L1 over-expressed Raji cells (FIG. 6B and 6D). At 40 uM, Isoliquiritigenin and licochalcone showed inhibition on NFAT activity of Jurkat cells in presence of SEE (FIG. 6B and 6D). Magnoflorine from Z showed inhibition on NFAT activity of Jurkat cells in presence of SEE (FIG. 6B and 6D).

### Example 5: The Action of YIV-906, S and its Components on NFAT Did Not Require TCRαβ

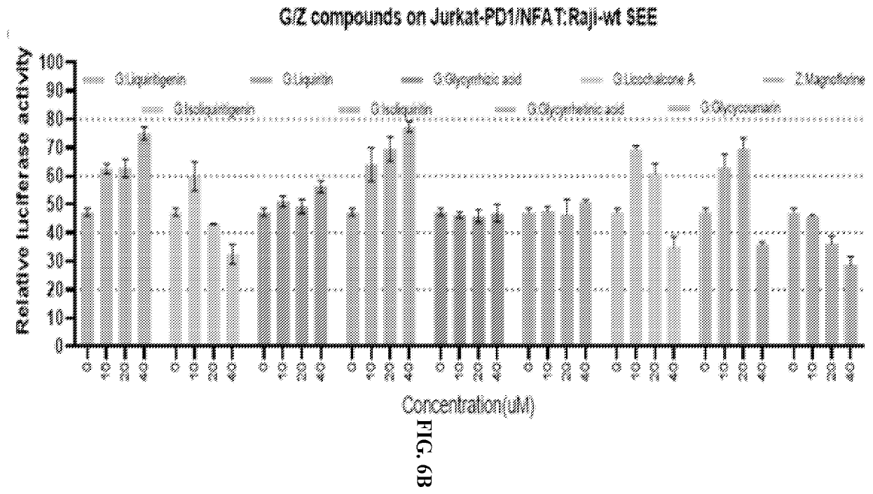

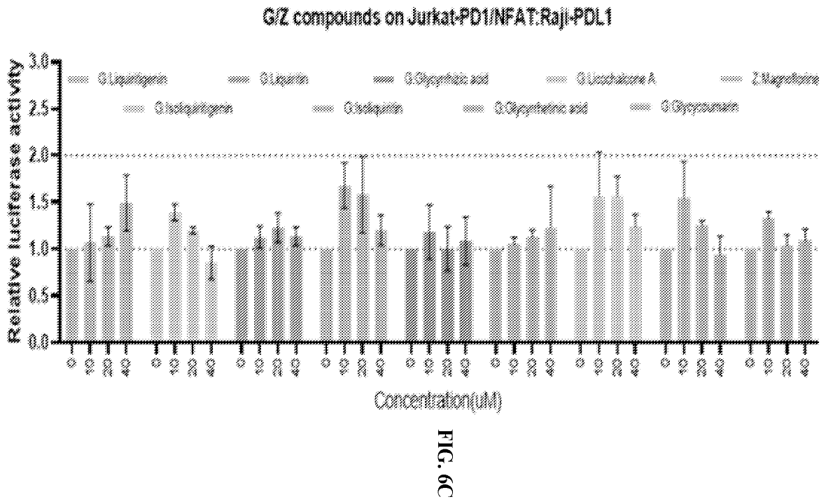

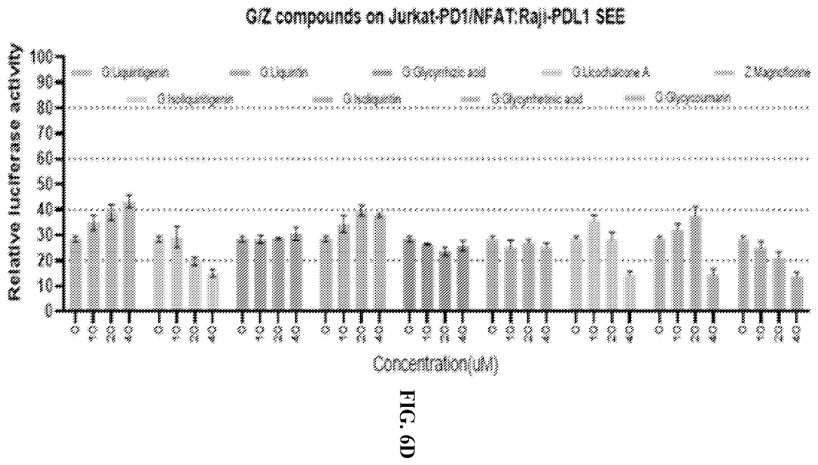

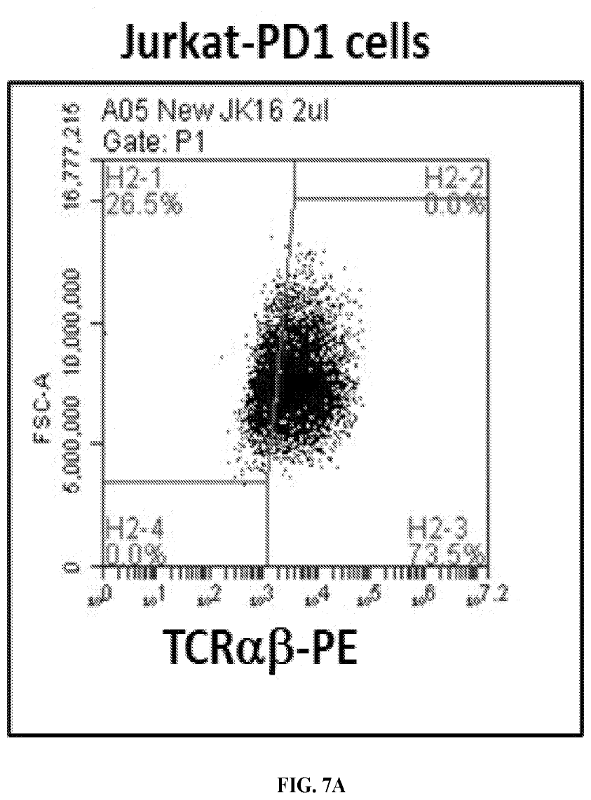

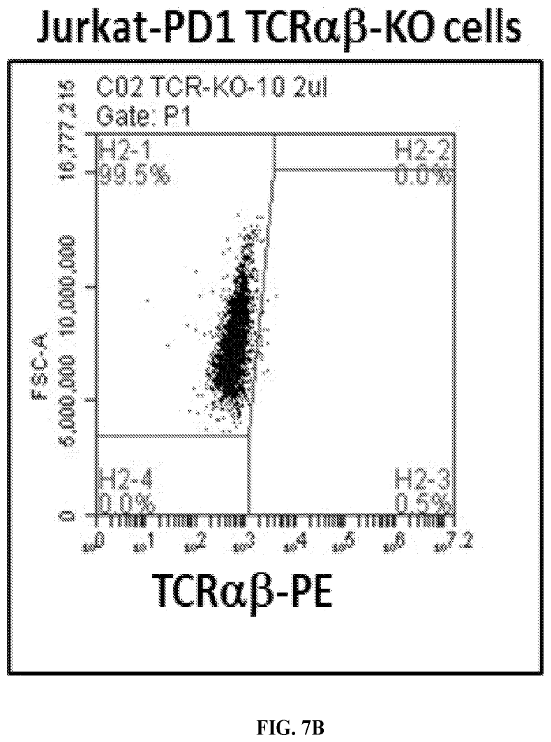

To investigate if YIV-906 and its components may directly activate TCR activity and then lead to NFAT activation, (KO) TCRαβ was knocked out from Jurkat-PD1 cells using CRISPR/Cas9 technology (FIGS. 7A and 7B). Jurkat-PD1 TCRαβ KO cells did not respond to SEE when they were co-cultured with wild type Raji cells. As shown in FIGS. 7C-7F, YIV-906, S and S compounds (oroxylin A 7-O-beta-D-glucuronide, baicalein, oroxylin A, and chrysin) had very similar impact on NFAT activity of Jurkat-PD1 and Jurkat-PD1 TCRαβ KO cells. The optimum dose for modulation could be difference for different compounds. These results suggested that the action of YIV-906 and its components on NFAT may bypass TCR.

### Example 6: YIV-906 and its Component Herbs Could Induce Protein Phosphorylation of T Cell Receptor Downstream Cascades

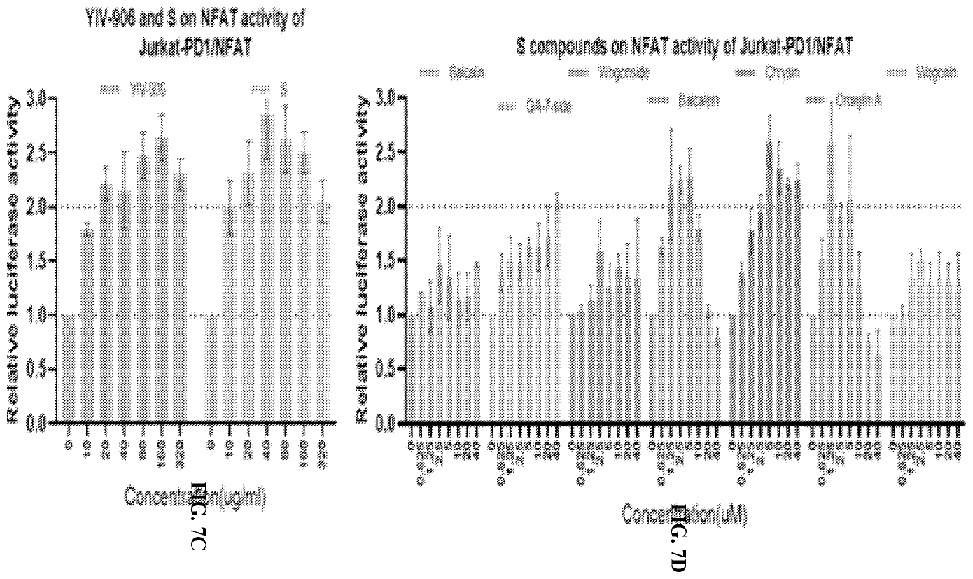

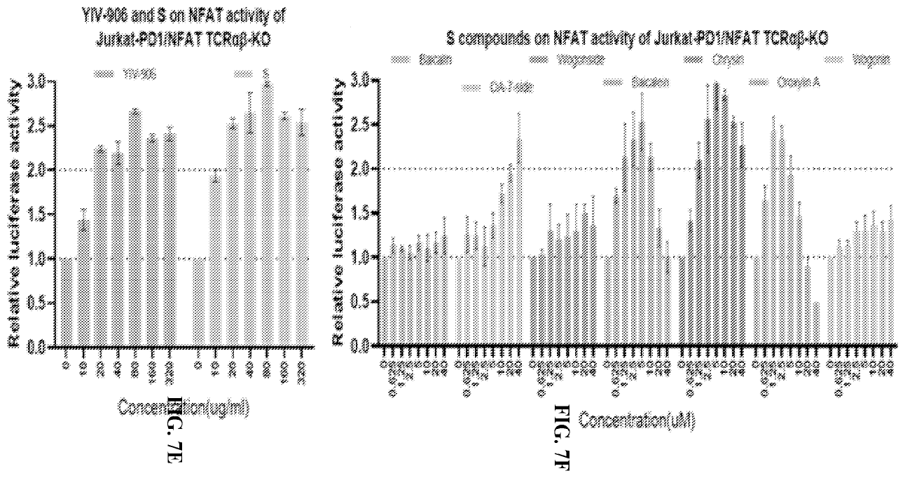

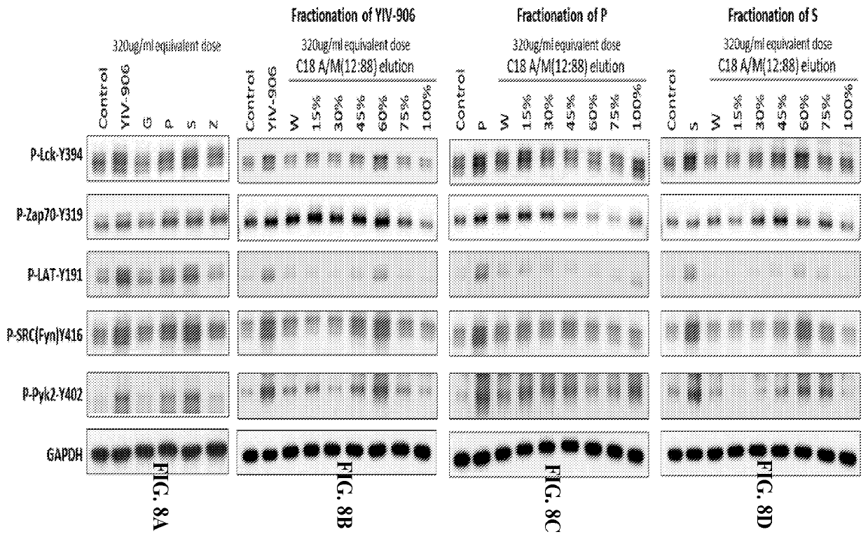

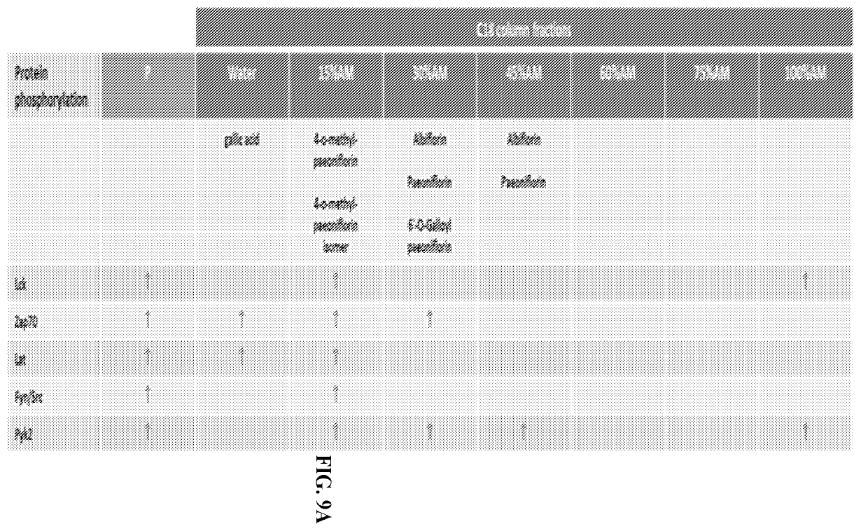

To test whether YIV-906 and its components may have direct impact on the downstream of TCR, the impact of YIV-906 and its components on the protein phosphorylation of T cell receptor downstream cascades including, Lck, Zap70, LAT, Fyn and Pyk2 of Jurkat cells was examined using western blot analysis. As shown in FIG. 8A, treatment of YIV-906, 320 ug/ml for 45 min, could induce phosphorylation of Lck, Zap70, LAT, Fyn and Pyk2. By comparing single herb, at equivalent concentration 320 ug/ml, S and P of YIV-906 were found to play the major role for inducing the above protein phosphorylation. After fractionating YIV-906 using C18 column, relatively higher activity for inducing protein phosphorylation could be found in 60% and 15% acetonitrile/methanol (A/M) fractions (FIG. 8B). For P, 15% A/M fraction could induce all those proteins phosphorylation. Some activity for different protein phosphorylation may be found in water, 30%, 45%, 100% A/M fraction of P (FIG. 8C). LCMS analysis revealed 4-o-methyl-paeoniflorin or its isomer could exist in 15% A/M fraction of P (FIG. 9A). Chemical present in water, 30%, 45%, 100% A/M fractions of P could also contribute to specific protein phosphorylation as indicated as red upward arrow (FIG. 9A). For S, major protein phosphorylation activity was found in 45%, 60% and 75% A/M fractions with some degree selectivity (FIG. 8D). 45% A/M fraction of S had stronger effect on P-Zap70-Y319 while 60% A/M fraction of S had stronger effect on P-Lck-Y39, P-LAT-Y191, P-SRC (Fyn)-Y416 and P-Pyk2-Y402 (FIG. 8D). Chemicals existed in these S fractionsare listed in FIG. 9B and those chemicals may contribute to specific protein phosphorylation as indicated asupward arrow in FIG. 9B. These chemicals of P and S may have different selectivity on their targets.

### Example 7: S and P Compounds Could Induce Protein Phosphorylation of T Cell Receptor Downstream Cascades

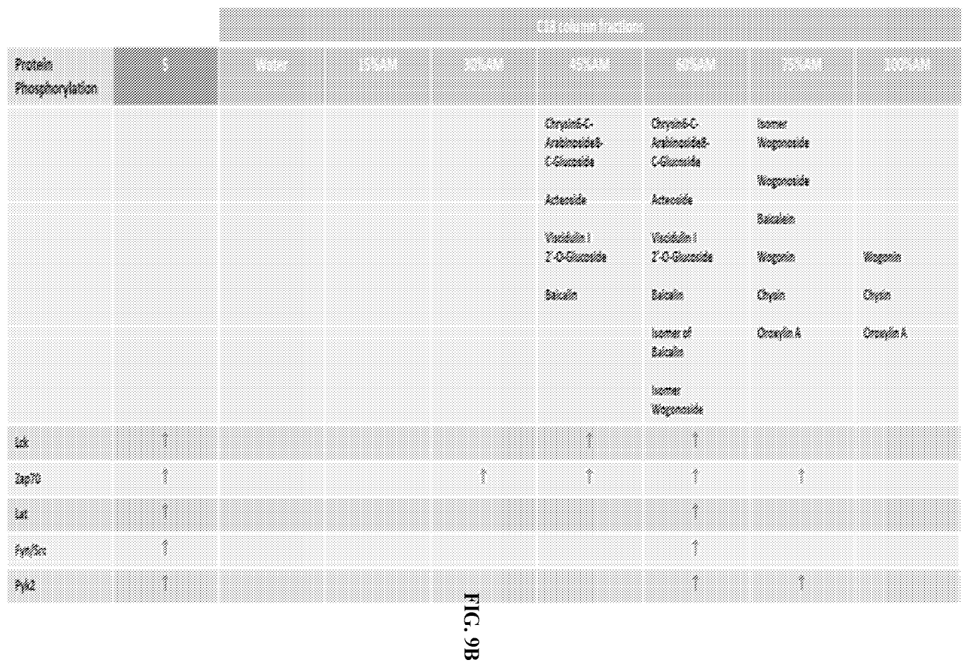

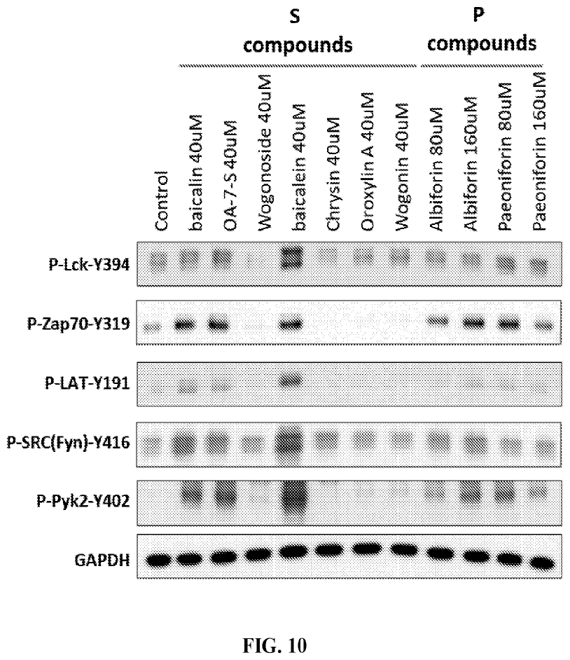

Some chemicals from S or P were examined for their ability to induce the protein phosphorylation of T cell receptor downstream cascades including, Lck, Zap70, LAT, Fyn and Pyk2 of Jurkat cells using western blot analysis. Baicalein, at 40 uM, could strongly induce protein phosphorylation of all examined proteins (FIG. 10). Baicalin and oroxylin A 7-O-beta-D-glucuronide (OA-7-S), at 40 uM, showed some induction effect on P-Zap70-Y319, P-SRC (Fyn)-Y416 and P-Pyk2-Y402 (FIG. 10). Albiforin and Paeoniforin had some effect on inducing P-Zap70-Y319 and P-Pyk2-Y402 (FIG. 10). These compounds had potential to activate T cell via stimulating downstream cascades of TCR.

### Example 8: YIV-906 Could Modulate SHP2 Activity of T Cells

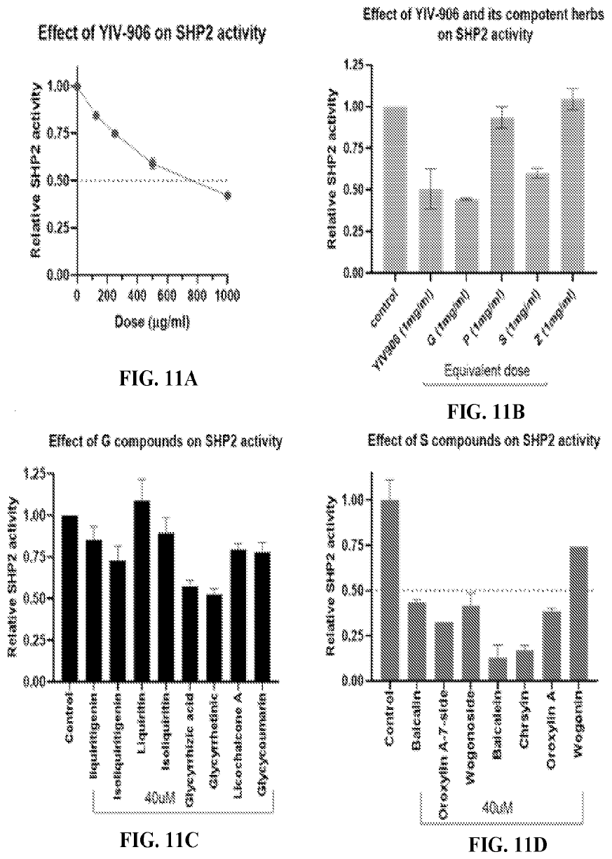

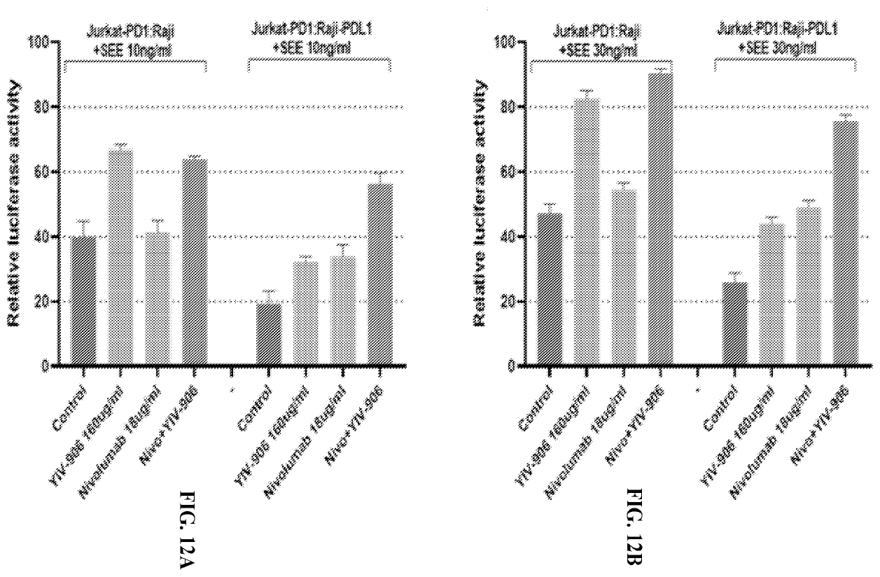

SHP2 (SH2-containing phosphatase 2, a protein tyrosine phosphatase) is an immediate downstream of PD1. Once PD1 binds to PD-L1, PD-1 phosphorylates its immune receptor tyrosine-based inhibitory motif (ITIM) and immune receptor tyrosine-based switch motif (ITSM) for recruiting and activating SHP2. Activated SHP2 initiates T cell inactivation by dephosphorylating TCR downstream cascades proteins such as Zap70. Inhibitors have potential to promote T cell activation and improve cancer treatment by blocking the PD1 action. As shown in FIG. 11A, YIV-906 showed inhibitory effect on SHP2 enzyme activity. G and S of YIV-906 were found to be the key herbs responsible for SHP2 inhibition (FIG. 11B). Glycyrrhizic acid and glycyrrhetinic acid from G had relatively higher inhibitory effects on SHP2 than other G compounds (FIG. 11C). Baicalin, oroxylin A 7-O-beta-D-glucuronide, wogonoside, baicalein, chrysin, oroxylin A from S showed inhibitory effect on SHP2 (FIG. 11D).

### Example 9: YIV-906 Could Further Enhance Anti-PD1 Antibody Action to Promote NFAT Activity of T Cells

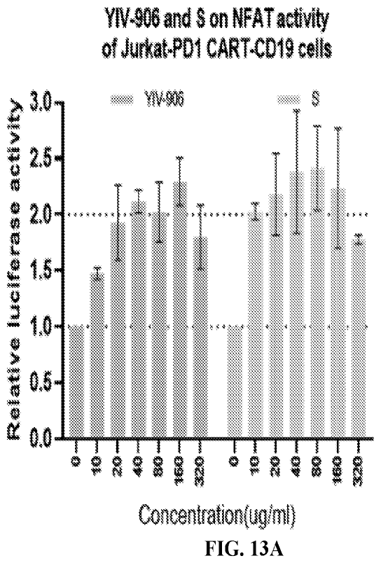

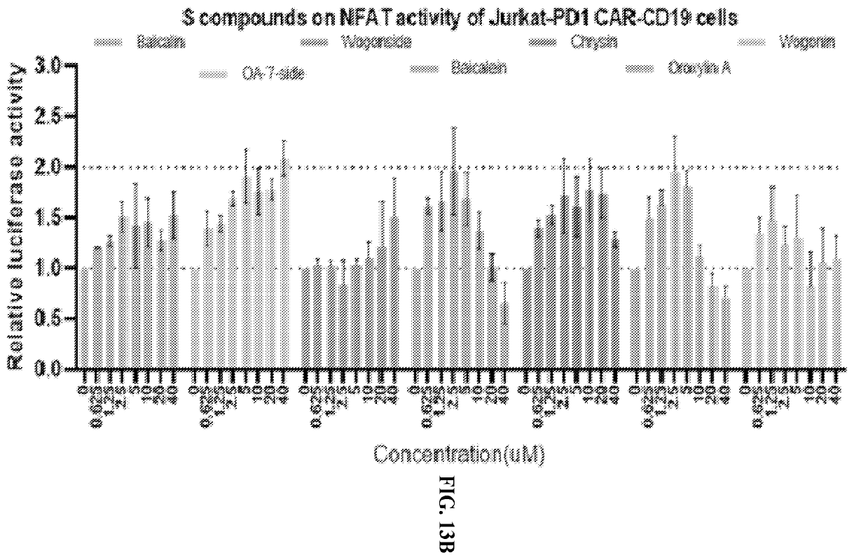

As YIV-906 could enhance the anti-tumor activity of anti-PD1 against tumor in animal models, it was analyzed whether YIV-906 can co-operate with anti-PD1 to promote NFAT activity for T cell activation. As shown earlier, co-culture Jurkat-PD1 cells and Raji-PD-L1 cells with SEE 10 ng/ml or 30 ng/ml inhibited NFAT activity of Jurkat cells by 50% as compared to NFAT activity shown by Jurkat-PD1 cells and wild type Raji cells. YIV-906 and Anti-PD1 (antibody produced from Nivolumab clone) rescued the depressed NFAT activity (due to the interaction of PD1 and PD-L1) in Jurkat-PD1 cells (FIG. 12A and 12B). In combination, YIV-906 did not comprise the action of Anti-PD1 while YIV-906 and Anti-PD1 induce stronger NFAT activity of Jurkat PD1 cells (FIG. 12A and 12B). Thus, YIV-906 has a potential to enhance immune checkpoint blockade therapy for treatment of cancer.

### Example 10: YIV-906 Could Modulate Chimeric Antigen Receptors Triggered NFAT Activity of T Cells

It is well known that MHC-antigen-TCR interaction will recruit CD3 receptors to transduce signals to NFAT. Chimeric antigen receptors (CARs) are composed of an antigen recognition domain, an extracellular hinge region, a transmembrane domain, and co-stimulatory domains such as immunoreceptor tyrosine-based activation motifs (ITAM) of CD3ζ((zeta). Once chimeric antigen receptor (CAR) binds to its target, a signal is transduced to ITAM of CD3ζ which further stimulates its downstream cascade leading to NFAT activation. Since MHC-antigen-TCR interaction or CAR-target interaction share the same downstream cascades for NFAT activation, and YIV-906 had potential to enhance MHC-antigen-TCR trigger NFAT response, it was questioned if YIV-906 can also modulate CAR triggered NFAT activity. Jurkat-PD1 NFAT luciferase receptor cells were stably transfected with CAR-CD19-CD3ζ(zeta) DNA which expresses CAR targeting CD19. NFAT activity could be stimulated by co-cultured Jurkat-PD1-CAR-CD19-CD3ζ and Raji which naturally expresses CD19 on cell surface.

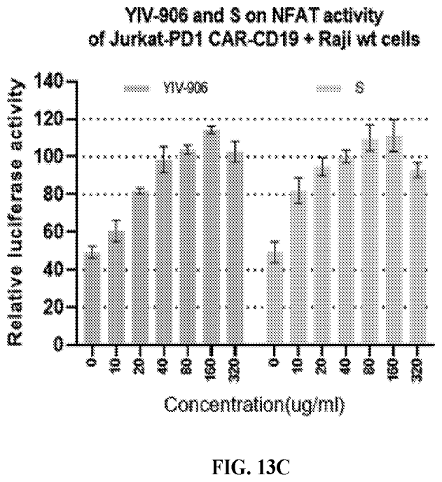

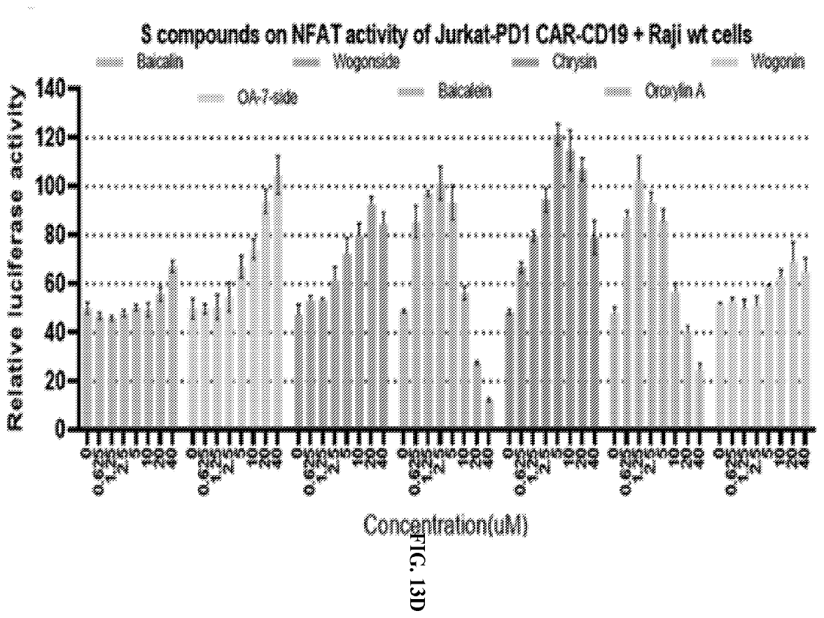

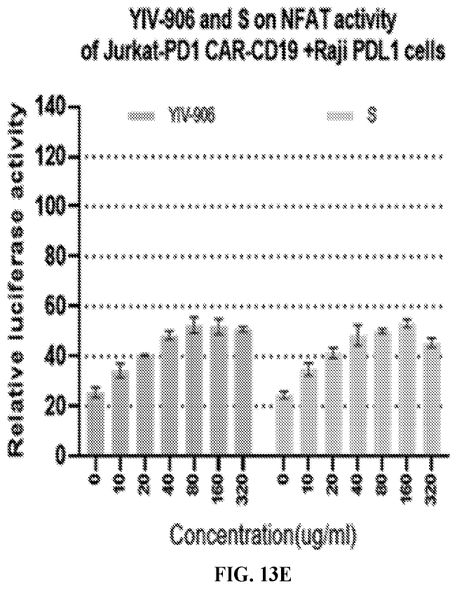

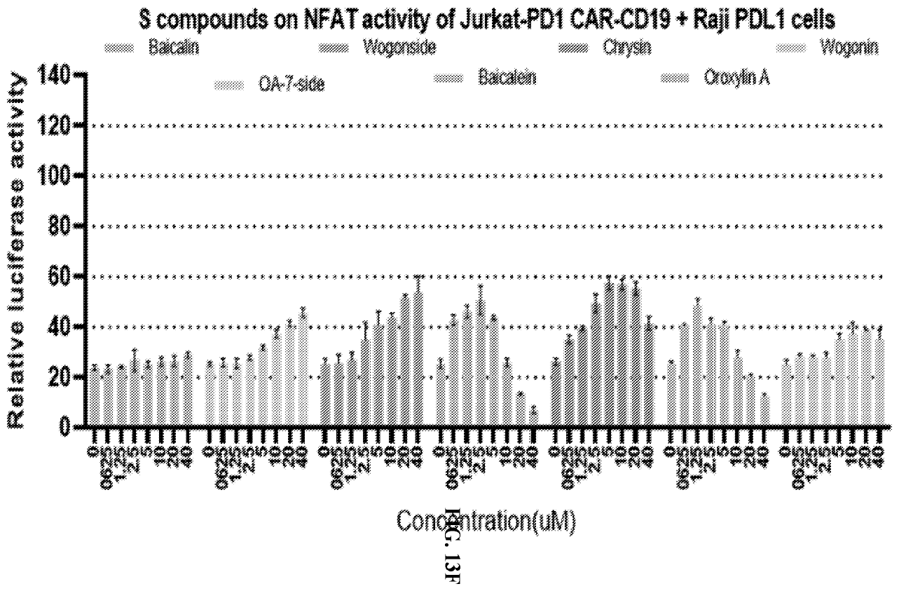

As shown in FIGS. 13A-13B, YIV-906, S and S compounds (oroxylin A 7-O-beta-D-glucuronide, baicalein, oroxylin A, and chrysin) had very similar impact on NFAT activity of Jurkat-PD1 CAR-CD19 cells and Jurkat-PD1 cells. When Jurkat-PD1 CAR-CD19 cells were co-cultured with Raji, NFAT activity could be induced about 40 folds. Under these conditions, YIV-906, S and S compounds (oroxylin A 7-O-beta-D-glucuronide, wogonoside, baicalein, oroxylin A, and chrysin) could modulate NFAT activity triggered by the interaction of chimeric antigen receptor (CAR-CD19-CD3ζ) on Jurkat cells and CD19 on Raji cells (FIGS. 13C-13D). PD1 and PD-L1 interaction can depress the NFAT activity triggered by the interaction of chimeric antigen receptor (CAR-CD19-CD3ζ) on Jurkat cells and CD19 on Raji cells (FIGS. 13E-13F) while YIV-906, S and S compounds (oroxylin A 7-O-beta-D-glucuronide, wogonoside, baicalein, oroxylin A, and chrysin) could restore this depressed NFAT activity (FIG. 13F). The optimum dose for modulation could be different for different compounds. These results suggested that YIV-906 and its components have potential to facilitate CAR T cell therapy for cancer treatment.

### Example 11

In summary, YIV-906 could modulate NFAT activity of T cells by inhibiting SHP2 and inducing TCR downstream protein phosphorylation. Multiple herbs: G, P, S and their compounds are involved, and they may have different targets with different potency. It is shown here that YIV-906 and some of its components could be used to modulate T cell activation for facilitating immune checkpoint blockade therapy or CAR T-cell therapy for treatment of cancer in patients. Since T cell activation is a very critical step for vaccines to trigger immune responses, YIV-906 and some of its components could also serve as adjuvant to enhance the effectiveness of vaccination.

### Enumerated Embodiments

The following enumerated embodiments are provided, the numbering of which is not to be construed as designating levels of importance.

Embodiment 1 provides a method of enhancing Nuclear Factor of Activated T-cells (NFAT) activity and/or restoring the suppressed NFAT activity in a subject in need thereof, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof,
(e) an herbal extract comprising an extract of G, a fraction thereof or any active chemical present in the herbal extract or the fraction thereof;
(f) an herbal extract of P, a fraction thereof or any active chemical present in the herbal extract or the fraction thereof, and
(g) an herbal extract comprising an extract of P, a fraction thereof or any active chemical present in the herbal extract or the fraction thereof.

Embodiment 2 provides the method of embodiment 1, wherein the active chemical in the herbal extract of S or the fraction thereof is at least one selected from the group consisting of oroxylin A 7-O-beta-D-glucuronide, baicalein, oroxylin A, baicalin, isomer of baicalin, chrysin 6-C-arabinoside 8-C-glucoside, acteoside, viscidulin I 2′-O-glucoside, and chrysin.

Embodiment 3 provides the method of embodiments 1-2, wherein the active chemical in the herbal extract of G or the fraction thereof is at least one selected from the group consisting of liquiritigenin, isoliquiritigenin, liquiritin, isoliquiritin, licochalcone A, and glycycoumarin.

Embodiment 4 provides the method of embodiments 1-3, wherein the active chemical in the herbal extract of P or the fraction thereof is at least one selected from the group consisting of gallic acid, 4-o-methyl paeoniflorin, 4-o-methyl paeoniflorin isomer, 6′-O-galloyl paconiflorin, albiflorin, and paeoniflorin.

Embodiment 5 provides the method of embodiments 1-4, wherein the herbal composition is administered to the subject orally.

Embodiment 6 provides the method of embodiments 1-5, wherein the herbal composition is formulated as one selected from the group consisting of a pill, a tablet, a capsule, soup, tea, a concentrate, a dragee, a liquid, a drop, and a gelcap.

Embodiment 7 provides the method of embodiments 1-6, wherein the therapeutically effective amount of the herbal composition is about 20 mg/day to about 2 g/day.

Embodiment 8 provides the method of embodiments 1-7, wherein the therapeutically effective amount of the herbal composition is about 1,600 mg/day.

Embodiment 9 provides the method of embodiments 1-8, wherein the method induces protein phosphorylation of T cell receptor (TCR) downstream cascades and inhibits SH2-containing phosphatase 2 (SHP2) activity.

Embodiment 10 provides the method of embodiments 1-9, wherein the herbal composition is administered at a time selected from prior to, simultaneously with, and after administration of an immune checkpoint inhibitor therapy or a Chimeric Antigen Receptor T(CAR T) cell therapy or a vaccine.

Embodiment 11 provides the method of embodiments 1-10, wherein the immune checkpoint inhibitor therapy includes administering at least one immune checkpoint inhibitor selected from the group consisting of Ipilimumab, Pembrolizumab, Nivolumab, Durvalumab, Avelumab, Dostarlimab-gxly, Cemiplimab-rwlc, Toripalimab, Camrelizumab, Tislelizumab, Penpulimab, Zimberelimab, Sintilimab, and Atezolizumab.

Embodiment 12 provides the method of embodiments 1-11, wherein the method is advantageous for at least one selected from the group consisting of:
a) enhancing efficacy of the CAR T cell therapy,
b) enhancing efficacy of the immune checkpoint inhibitor therapy, and
c) enhancing effectiveness of the vaccine.

Embodiment 13 provides the method of embodiments 1-12, wherein the method is used for cancer chemoprevention.

Embodiment 14 provides a method of enhancing efficacy of Chimeric Antigen Receptor T-cell (CAR T) immunotherapy in a subject, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof.
(f) an herbal extract of P, a fraction thereof or any active chemical present in the herbal extract or the fraction thereof, and
(g) an herbal extract comprising an extract of P, a fraction thereof or any active chemical present in the herbal extract or the fraction thereof.

Embodiment 15 provides a method of enhancing efficacy of immune checkpoint blockade therapy in a subject, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof.
(f) an herbal extract of P, a fraction thereof or any active chemical present in the herbal extract or the fraction thereof, and
(g) an herbal extract comprising an extract of P, a fraction thereof or any active chemical present in the herbal extract or the fraction thereof.

Embodiment 16 provides a method of enhancing effectiveness of a vaccine in a subject, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof;
(f) an herbal extract of P, a fraction thereof or any active chemical present in the herbal extract or the fraction thereof; and
(g) an herbal extract comprising an extract of P, a fraction thereof or any active chemical present in the herbal extract or the fraction thereof.

Embodiment 17 provides the method of embodiments 1-16, wherein the subject is a mammal.

Embodiment 18 provides the method of embodiments 1-17, wherein the subject is a human subject.

Embodiment 19 provides an herbal composition for modulating NFAT activity in T cells of a subject in need thereof, wherein the composition comprises at least one selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or fraction thereof;
(c) an herbal extract comprising an extract of S, a fraction thereof or any active chemical present in the herbal extract or fraction thereof;
(d) an herbal extract of G, a fraction thereof or any active chemical present in the herbal extract or fraction thereof;
(e) an herbal extract comprising an extract of G, a fraction thereof or any active chemical present in the herbal extract or fraction thereof;
(f) an herbal extract of P, a fraction thereof or any active chemical present in the herbal extract or fraction thereof;
(g) an herbal extract comprising an extract of P, a fraction thereof or any active chemical present in the herbal extract or fraction thereof;
(h) an herbal extract of Z, a fraction thereof or any active chemical present in the herbal extract or fraction thereof; and
(i) an herbal extract comprising an extract of Z, a fraction thereof or any active chemical present in the herbal extract or fraction thereof.

Embodiment 20 provides the composition of embodiment 19, wherein the herbal composition is administered to the subject orally.

Embodiment 21 provides the composition of embodiments 19-20, wherein the herbal composition is formulated as one selected from the group consisting of a pill, a tablet, a capsule, a soup, a tea, a concentrate, a dragee, a liquid, a drop, and a gelcap.

Embodiment 22 provides the composition of embodiments 19-21, wherein the composition further comprises a pharmaceutically acceptable carrier.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure is made with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of enhancing Nuclear Factor of Activated T-cells (NFAT) activity and/or restoring the suppressed NFAT activity in a subject in need thereof, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof;
(f) an herbal extract of P, a fraction thereof or any active chemical present in the herbal extract or the fraction thereof; and
(g) an herbal extract comprising an extract of P, a fraction thereof or any active chemical present in the herbal extract or the fraction thereof.

2. The method of claim 1, wherein the active chemical in the herbal extract of S or the fraction thereof is at least one selected from the group consisting of oroxylin A 7-O-beta-D-glucuronide, baicalein, oroxylin A, baicalin, isomer of baicalin, chrysin 6-C-arabinoside 8-C-glucoside, acteoside, viscidulin I 2′-O-glucoside, and chrysin.

3. The method of claim 1, wherein the active chemical in the herbal extract of G or the fraction thereof is at least one selected from the group consisting of liquiritigenin, isoliquiritigenin, liquiritin, isoliquiritin, licochalcone A, and glycycoumarin.

4. The method of claim 1, wherein the active chemical in the herbal extract of P or the fraction thereof is at least one selected from the group consisting of gallic acid, 4-o-methyl paeoniflorin, 4-o-methyl paeoniflorin isomer, 6′-O-galloyl paeoniflorin, albiflorin, and paeoniflorin.

5. The method of claim 1, wherein the herbal composition is administered to the subject orally.

6. The method of claim 1, wherein the herbal composition is formulated as one selected from the group consisting of a pill, a tablet, a capsule, a soup, a tea, a concentrate, a dragee, a liquid, a drop, and a gelcap.

7. The method of claim 1, wherein the therapeutically effective amount of the herbal composition is about 20 mg/day to about 2 g/day.

8. The method of claim 7, wherein the therapeutically effective amount of the herbal composition is about 1,600 mg/day.

9. The method of claim 1, wherein the method induces protein phosphorylation of T cell receptor (TCR) downstream cascades and inhibits SH2-containing phosphatase 2 (SHP2) activity.

10. The method of claim 1, wherein the herbal composition is administered at a time selected from prior to, simultaneously with, and after administration of an immune checkpoint inhibitor therapy or a Chimeric Antigen Receptor T(CAR T) cell therapy or a vaccine.

11. The method of claim 10, wherein the immune checkpoint inhibitor therapy includes administering at least one immune checkpoint inhibitor selected from the group consisting of Ipilimumab, Pembrolizumab, Nivolumab, Durvalumab, Avelumab, Dostarlimab-gxly, Cemiplimab-rwlc, Toripalimab, Camrelizumab, Tislelizumab, Penpulimab, Zimberelimab, Sintilimab, and Atezolizumab.

12. The method of claim 10, wherein the method is advantageous for at least one selected from the group consisting of:
a) enhancing efficacy of the CAR T cell therapy,
b) enhancing efficacy of the immune checkpoint inhibitor therapy, and
c) enhancing effectiveness of the vaccine.

13. The method of claim 1, wherein the method is used for cancer chemoprevention.

14. A method of enhancing efficacy of Chimeric Antigen Receptor T-cell (CAR T) immunotherapy in a subject, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof.
(f) an herbal extract of P, a fraction thereof or any active chemical present in the herbal extract or the fraction thereof; and
(g) an herbal extract comprising an extract of P, a fraction thereof or any active chemical present in the herbal extract or the fraction thereof.

15. A method of enhancing efficacy of immune checkpoint blockade therapy in a subject, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof.
(f) an herbal extract of P, a fraction thereof or any active chemical present in the herbal extract or the fraction thereof; and
(g) an herbal extract comprising an extract of P, a fraction thereof or any active chemical present in the herbal extract or the fraction thereof.

16. A method of enhancing effectiveness of a vaccine in a subject, wherein the method comprises administering to the subject a therapeutically effective amount of least one herbal composition selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or the fraction thereof;
(c) an herbal extract comprising an extract of S, a fraction thereof or any active chemical present in the herbal extract or the fraction thereof;
(d) an herbal extract of G, a fraction thereof or any active chemical present in the herbal extract or the fraction thereof;
(e) an herbal extract comprising an extract of G, a fraction thereof or any active chemical present in the herbal extract or the fraction thereof;
(f) an herbal extract of P, a fraction thereof or any active chemical present in the herbal extract or the fraction thereof; and
(g) an herbal extract comprising an extract of P, a fraction thereof or any active chemical present in the herbal extract or the fraction thereof.

17. The method of claim 1, wherein the subject is a mammal.

18. The method of claim 17, wherein the subject is a human subject.

19. An herbal composition for modulating NFAT activity in T cells of a subject in need thereof, wherein the composition comprises at least one selected from the group consisting of:
(a) an herbal extract PHY906, which comprises herbal extracts of Scutellaria baicalensis (S), Glycyrrhiza uralensis (G), Paeonia lactiflora (P), and Ziziphus jujuba (Z), a fraction thereof or any active chemical present in the herbal extract or fraction thereof;
(b) an herbal extract of S, a fraction thereof or any active chemical present in the herbal extract or fraction thereof;
(c) an herbal extract comprising an extract of S, a fraction thereof or any active chemical present in the herbal extract or fraction thereof;
(d) an herbal extract of G, a fraction thereof or any active chemical present in the herbal extract or fraction thereof;
(e) an herbal extract comprising an extract of G, a fraction thereof or any active chemical present in the herbal extract or fraction thereof;
(f) an herbal extract of P, a fraction thereof or any active chemical present in the herbal extract or fraction thereof;
(g) an herbal extract comprising an extract of P, a fraction thereof or any active chemical present in the herbal extract or fraction thereof;
(h) an herbal extract of Z, a fraction thereof or any active chemical present in the herbal extract or fraction thereof; and
(i) an herbal extract comprising an extract of Z, a fraction thereof or any active chemical present in the herbal extract or fraction thereof.

20. The composition of claim 19, wherein the herbal composition is administered to the subject orally.

21. The composition of claim 19, wherein the herbal composition is formulated as one selected from the group consisting of a pill, a tablet, a capsule, a soup, a tea, a concentrate, a dragee, a liquid, a drop, and a gelcap.

22. The composition of claim 19, wherein the composition further comprises a pharmaceutically acceptable carrier.
